(19) United States
(12) Patent Application Publication
(10) Pub. No.: US 2008/0227219 A1
(43) Pub. Date: Sep. 18, 2008

(54) Electrochemiluminescent assay

(76) Inventor: Frank Gamez, Silver Spring, MD (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/274,305

(22) Filed: Nov. 16, 2005

Related U.S. Application Data
(60) Provisional application No. 60/628,122, filed on Nov. 17, 2004.

(51) Int. Cl. G01N 33/543

(52) U.S. Cl. 436/518

(57) ABSTRACT

Disclosed herein are compositions that may be used in an assay, such as an immunoassay, for detecting and/or quantifying at least one analyte of interest, such as an antigen. Also disclosed are control/calibrator compositions and methods for preparing control/calibrator compositions that may be used in assays, such as immunoassays, methods for detecting and/or quantifying an analyte with the compositions, and kits containing the compositions.

### FIELD OF THE INVENTION

The present invention relates to assay compositions, methods, and kits comprising an assay for detection of at least one analyte, such as an antigen or hapten in a sample. The analyte(s) can be provided, for example, in a biological sample.

### BACKGROUND

In the medical, environmental, biodefense, and food safety communities, immunodiagnostic testing can provide a simple assessment and rapid identification of diseases and contaminants that are harmful to society. To prevent the occurrence of protracted illness and/or endemic disease, there is a need for simple confirmatory assays that provide qualitative, semi-quantitative, and quantitative assessment for the detection of analytes, such as an antigen in a clinical specimen, soil or water sample, or food. In addition, due to the realization of the threat of national terrorism in recent years, many diagnostic tests are designed to be performed at satellite sites other than established laboratories.

Conventional immunoassay-based detection systems rely upon an antibody-antigen interaction, which requires the addition of multiple assay components in a sequential manner to produce a detectable event. Although generally reliable for positive identification, these assay and reagent preparation procedures can be involved and time consuming. One drawback associated with present procedures is that the sequential addition and transfer of multiple reagents is necessary to perform an assay. Each additional step for a detection assay may increase the degree of difficulty for execution by the operator. Consequently, the assay may be prone to mistake, often resulting in a higher margin for error.

Accordingly, there remains a need to develop new methods and reagents for reliable and easy to use diagnostic assays that may be performed by non-technical or lay personnel.

### SUMMARY

Disclosed herein are dry compositions that can, for example, be used in assays. The dry composition can comprise two or more reagents or assays components, such as labeled binding partners for specifically binding to an analyte and/or positive control/calibrator reagents, such that the dry composition can function as a single reagent. By providing these reagents in a dry composition, an analyst may minimize the number of steps carried out when performing the assay by potentially avoiding sequential addition and transfer of multiple reagents typically necessary to perform an assay.

In some embodiments, the invention provides a dry composition for use as an assay positive control/calibrator comprising:

(a) a labeled binding partner comprising a label and a binding partner wherein said labeled binding partner can specifically bind to an analyte; and

(b) a positive control/calibrator reagent comprising a known amount of the analyte or an analog of the analyte that can specifically bind to the binding partner;

wherein the composition has a moisture content of less than or equal to about 5% by weight, relative to the total weight of the composition.

In certain embodiments, the invention provides a dry composition for use as an assay positive control/calibrator comprising:

(a) a first binding partner for specifically binding an analyte;

(b) a support for binding the first binding partner without blocking the binding of the analyte;

(c) a labeled second binding partner comprising a label and a binding partner wherein said labeled second binding partner can specifically bind to the same analyte; and

(b) a positive control/calibrator reagent comprising a known amount of the analyte or an analog of the analyte that can specifically bind to both the first binding partner and the second binding partner;

wherein the composition has a moisture content of less than or equal to about 5% by weight, relative to the total weight of the composition.

In various embodiments, the invention provides a dry composition for use as an assay positive control/calibrator comprising:

(a) a first binding partner for specifically binding an analyte;

(b) the labeled analyte or a labeled analog of the analyte, comprising a label and the analyte or an analog of the analyte, wherein said labeled analyte or labeled analog of the analyte competes with the analyte for binding to the first binding partner; and

(c) a positive control/calibrator reagent comprising a known amount of the analyte or an analog of the analyte that can specifically bind to the first binding partner;

wherein the composition has a moisture content of less than or equal to about 5% by weight, relative to the total weight of the composition.

In some embodiments, the invention provides a dry composition for use as an assay positive control/calibrator comprising:

(a) a first binding partner for specifically binding an analyte;

(b) a support that binds to the first binding partner without blocking the binding of the analyte.

(c) the labeled analyte or a labeled analog of the analyte, comprising a label and the analyte or an analog of the analyte, wherein said labeled analyte or analog of the analyte competes with the analyte in a sample for binding to the first binding partner; and

(d) a positive control/calibrator reagent comprising a known amount of the analyte or an analog of the analyte that can specifically bind to the first binding partner;

wherein the composition has a moisture content of less than or equal to about 5% by weight, relative to the total weight of the composition.

In various embodiments, the invention provides a method for detecting and/or quantifying an analyte, comprising:

(a) providing a sample, which may contain the analyte;

(b) forming a test reaction mixture by combining the sample with a composition comprising a labeled binding partner for specifically binding to the analyte;

(c) forming at least one positive control/calibrator reaction mixture by rehydrating at least one dry composition comprising:
(i) said labeled binding partner; and
(ii) a control/calibrator reagent comprising a known amount of the analyte or an analog of the analyte that can specifically bind to the binding partner;

(d) incubating the test reaction mixture and the at least one positive control/calibrator reaction mixture ; and

(e) measuring a signal attributable to a complex formed by binding the analyte to the labeled binding partner for each of the reaction mixtures.

In some embodiments, the invention provides a method for detecting and/or quantifying an analyte, comprising:

(a) providing a sample, which may contain the analyte;

(b) forming a test reaction mixture by combining the sample with a composition comprising:
(i) a first binding partner for specifically binding the analyte;
(ii) a support for binding the first binding partner without blocking the binding of the analyte; and
(iii) a labeled second binding partner for specifically binding to the same analyte;

(c) forming at least one positive control/calibrator reaction mixture by rehydrating at least one dry composition comprising:
(i) said first binding partner;
(ii) said support;
(iii) said labeled second binding partner; and
(iv) a positive control/calibrator reagent comprising a known amount of the analyte or an analog of the analyte that can specifically bind to both the first binding partner and the second binding partner;

(d) incubating the test reaction mixture and the at least one positive control/calibrator reaction mixture; and

(e) measuring a signal attributable to a complex formed by binding the analyte to the labeled binding partner for each of the reaction mixtures.

In various embodiments, the invention provides a method for detecting and/or quantifying an analyte, comprising:

(a) providing a sample, which may contain the analyte;

(b) forming a test reaction mixture by combining the sample with a composition comprising:
(i) a first binding partner for specifically binding the analyte;
(ii) a labeled analyte or analog of the analyte that competes with the analyte in the sample for binding to the first binding partner;

(c) forming at least one positive control/calibrator reaction mixture by rehydrating at least one dry composition comprising:
(i) said first binding partner;
(ii) said labeled analyte or analog of the analyte; and
(iii) a positive control/calibrator reagent comprising a known amount of the analyte or an analog of the analyte that can specifically bind to the first binding partner;

(d) incubating the test reaction mixture and the at least one positive control/calibrator reaction mixture; and

(e) measuring a signal attributable to a complex formed by binding the analyte to said first binding partner for each of the reaction mixtures.

In some embodiments, the invention provides a method for detecting and/or quantifying an analyte, comprising:

(a) providing a sample, which may contain the analyte;

(b) forming a test reaction mixture by combining the sample with a composition comprising:
(i) a first binding partner for specifically binding the analyte;
(ii) a support for binding the first binding partner without blocking the binding of the analyte
(iii) a labeled analyte or analog of the analyte that competes with the analyte in the sample for binding to the first binding partner;

(c) forming at least one positive control/calibrator reaction mixture by rehydrating at least one dry composition comprising:
(i) said first binding partner;
(ii) said support
(iii) said labeled analyte or analog of the analyte; and
(iv) a positive control/calibrator reagent comprising a known amount of the analyte or an analog of the analyte that can specifically bind to the first binding partner;

(d) incubating the test reaction mixture and the at least one positive control/calibrator reaction mixture;

(e) measuring a signal attributable to a complex formed by binding the analyte to said first binding partner for each of the reaction mixtures.

In some embodiments, the invention provides a kit comprising

(a) a dry composition comprising reagents used for a binding assay and a positive control/calibrator reagent;

(b) at least one container in which the dry composition is located; and

(c) calibration/control information, or a key for obtaining calibration/control information.

In certain embodiments, the invention provides a method of preparing a composition, comprising:

(a) preparing a first solution comprising a labeled binding partner;

(b) freezing the solution formed in (a);

(c) adding a second solution comprising a positive control/calibrator reagent to the frozen mixture at a temperature sufficient to freeze the second solution; and

(d) drying the first and second solutions.

In various embodiments, the invention provides a method of preparing a composition, comprising:

(a) preparing a first solution comprising a first binding partner for specifically binding an analyte and an assay buffer (b) freezing the mixture formed in (a);

(c) adding a second solution to the frozen mixture at a temperature sufficient to freeze the second solution, said second solution comprising a positive control/calibrator reagent and a labeled analyte or analog of the analyte that competes with the analyte in a sample for binding to the binding partner, and

(d) drying the first and second solution

In certain embodiments, the invention provides a method of preparing a composition, comprising:

(a) drying a first solution comprising a labeled binding partner;

(b) drying a second solution comprising a positive control/calibrator reagent; and

(c) combining the dried first and second solutions.

In some embodiments, the invention provides a method of preparing a composition, comprising:

(a) drying a first solution comprising a first binding partner for specifically binding an analyte and an assay buffer;

(b) drying a second solution comprising (i) a positive control/calibrator reagent and (ii) a labeled analyte or analog of the analyte that competes with the analyte in a sample for binding to the binding partner; and

(c) combining the dried first and second solutions.

It is to be understood that both the foregoing general description and the following detailed description are exemplary and explanatory only and are not restrictive of the invention, as claimed.

The accompanying drawings, which are incorporated in and constitute a part of this disclosure, illustrate embodiments of the invention.

### BRIEF DESCRIPTION OF THE DRAWINGS

FIG. 1 is a dose-response curve generated at various incubation time points for PSA calibrator solutions A-G;

FIG. 2 is a bar chart showing an ORIGEN® Analyzer assay drift over one carousel 30 min incubation;

FIG. 3 is a bar chart of Signal:Background ratios for PSA calibrator solutions A-G for all instruments;

FIG. 4 is a calibrator dose-response curve comparing the wet and dry reagent assays; and

FIG. 5 is a bar chart showing a comparison of dose-response curves for wet versus dry PSA calibrators A-G.

### DETAILED DESCRIPTION

Disclosed herein are compositions for use in an assay, such as an immunoassay, their use in methods for detecting and/or quantifying an analyte, and kits incorporating these compositions. The dry composition can comprise two or more reagents or assays components, such as labeled binding partners for specifically binding to an analyte and/or positive control/calibrator reagents, such that the dry composition can function as a single reagent. By providing these reagents in a dry composition, an analyst may minimize the number of steps carried out when performing the assay by potentially avoiding sequential addition and transfer of multiple reagents typically necessary to perform an assay.

U.S. Patent Application Publication No. 2003/0108973, the disclosure of which is incorporated herein by reference, discloses an immunoassay that minimizes the number of steps performed by a user. The '973 publication describes a reagent comprising (a) an immobilized capture antibody and (b) a labeled reporter antibody, wherein the immobilized capture antibody and the labeled reporter antibody bind specifically to the same analyte. This publication also describes the preparation of the reagent by drying a liquid comprising the labeled reporter antibody in the presence of the immobilized capture antibody. The assay can be performed by reconstituting the reagents with an antigen sample.

Although providing assay reagents as a single reagent may simplify the assay procedure, many variables, such as those arising from environmental conditions or impurities, can still affect the outcome of an assay. To account for these factors, it is common practice to incorporate controls in the assay to allow a user to assess the results. For example, where a certain outcome is expected, a positive control can be useful to indicate that the assay works for its intended purpose. In a more specific example, where the assay is used for determining the presence of, for example, a particular antigen, the sample to be tested can include a known amount of the antigen. This known amount can act as a standard against which to assess the sample. The absence of the antigen would still inform the user that the assay generated the expected result. Additionally, the positive control can be used to quantitatively assess the assay results. To the knowledge of the inventors, however, reagents for positive control/calibrators have previously been added as wet reagents.

Moreover, available methods do not permit the preparation of dried, premixed reagents for competitive binding assays. Such assays generally comprise at least one binding partner, e.g., an antibody that binds the analyte of interest and a competitor that also binds to the binding partner, e.g., an analog of the analyte of interest or a known amount of the analyte itself. If these components are premixed before the sample is added, the competitor may bind to the binding partner. When the dried reagents are reconstituted by adding liquid sample, the rate at which the reaction reaches equilibrium will be perturbed by the prebinding.

### Definitions

In order to more clearly understand the invention, certain terms are defined as follows.

The term “dry composition,” as used herein, means that the composition has a moisture content of less than or equal to about 5% by weight, relative to the total weight of the composition. Examples of dry compositions include compositions that have a moisture content of less than or equal to about 3% by weight relative to the total weight of the composition, compositions that have a moisture content of less than or equal to about 1% by weight relative to the total weight of the composition, and compositions that have a moisture content ranging from about 1% to about 3% by weight, relative to the total weight of the composition.

The term “binding partner,” as used herein, means a substance that can bind specifically to an analyte of interest. In general, specific binding is characterized by a relatively high affinity and a relatively low to moderate capacity. Nonspecific binding usually has a low affinity with a moderate to high capacity. Typically, binding is considered specific when the affinity constant Ka is higher than about 10⁶ M⁻¹, or is higher than about 10⁸ M⁻¹. A higher affinity constant indicates greater affinity, and thus typically greater specificity. For example, antibodies typically bind antigens with an affinity constant in the range of 10⁶ M⁻¹ to 10⁹ M¹ or higher.

Examples of binding partners include complementary nucleic acid sequences (e.g., two DNA sequences which hybridize to each other; two RNA sequences which hybridize to each other; a DNA and an RNA sequence which hybridize to each other), an antibody and an antigen, a receptor and a ligand (e.g., TNF and TNFr-I, CD142 and Factor VIIa, B7-2 and CD28, HIV-1 and CD4, ATR/TEM8 or CMG and the protective antigen moiety of anthrax toxin), an enzyme and a substrate, or a molecule and a binding protein (e.g., vitamin B12 and intrinsic factor, folate and folate binding protein).

Further examples of binding partners include antibodies. The term “antibody,” as used herein, means an immunoglobulin or a part thereof, and encompasses any polypeptide comprising an antigen-binding site regardless of the source, method of production, or other characteristics. The term includes, for example, polyclonal, monoclonal, monospecific, polyspecific, humanized, single-chain, chimeric, synthetic, recombinant, hybrid, mutated, and CDR-grafted antibodies as well as fusion proteins. A part of an antibody can include any fragment which can bind antigen, for example, Fab, Fab′, F(ab′)₂, Facb, Fv, ScFv, Fd, V_{H}, and V_{L}.

Further examples of binding partners include monoclonal antibodies. A large number of monoclonal antibodies that bind to various analytes of interest are available, as exemplified by the listings in various catalogs, such as: Biochemicals and Reagents for Life Science Research, Sigma-Aldrich Co., P.O. Box 14508, St. Louis, Mo., 63178, 1999; the Life Technologies Catalog, Life Technologies, Gaithersburg, Md.; and the Pierce Catalog, Pierce Chemical Company, P.O. Box 1 17, Rockford, Ill. 61105, 1994, the disclosures of which are incorporated herein by reference.

Other exemplary monoclonal antibodies include those that bind specifically to β-actin, DNA, digoxin, insulin, progesterone, human leukocyte markers, human interleukin-10, human interferon, human fibrinogen, p53, hepatitis B virus or a portion thereof, HIV virus or a portion thereof, tumor necrosis factor, and FK-506. In certain embodiments, the monoclonal antibody is chosen from antibodies that bind specifically to at least one of T4, T3, free T3, free T4, TSH (thyroid-stimulating hormone), thyroglobulin, TSH receptor, prolactin, LH (luteinizing hormone), FSH (follicle stimulating hormone), testosterone, progesterone, estradiol, hCG (human Chorionic Gondaotropin), hCG+β, SHBG (sex hormone-binding globulin), DHEA-S (dehydroepiandrosterone sulfate), hGH (human growth hormone), ACTH (adrenocorticotropic hormone), cortisol, insulin, ferritin, folate, RBC (red blood cell) folate, vitamin B12, vitamin D, C-peptide, troponin T, CK-MB (creatine kinase-myoglobin), myoglobin, pro-BNP (brain natriuretic peptide), HbsAg (hepatitis B surface antigen), HbeAg (hepatitis B e antigen), HIV antigen, HIV combined, H. pylori, β-cROSSLAPS, osteocalcin, PTH (parathyroid hormone), IgE, digoxin, digitoxin, AFP (α-fetoprotein), CEA (carcinoembryonic antigen), PSA (prostate specific antigen), free PSA, CA (cancer antigen) 19-9, CA 12-5, CA 72-4, cyfra 21-1, NSE (neuron specific enolase), S 100, P1NP (procollagen type 1 N-propeptide), PAPP-A (pregnancy-associated plasma protein-A), Lp-PLA2 (lipoprotein-associated phospholipase A2), sCD40L (soluble CD40 Ligand), IL 18, and Survivin.

Other exemplary monoclonal antibodies include anti-TPO (antithyroid peroxidase antibody), anti-HBc (Hepatitis B c antigen), anti-HBc/IgM, anti-HAV (hepatitis A virus), anti-HAV/IgM, anti-HCV (hepatitis C virus), anti-HIV, anti-HIV p-24, anti-rubella IgG, anti-rubella IgM, anti-toxoplasmosis IgG, anti-toxoplasmosis IgM, anti-CMV (cytomegalovirus) IgG, anti-CMV IgM, anti-HGV (hepatitis G virus), and anti-HTLV (human T-lymphotropic virus).

Further examples of binding partners include binding proteins, for example, vitamin B12 binding protein; DNA binding proteins such as the superclasses of basic domains, zinc-coordinating DNA binding domains, Helix-turn-helix, beta scaffold factors with minor groove contacts, and other transcription factors that are not antibodies.

The term “labeled binding partner,” as used herein, means a binding partner that is labeled with an atom, moiety, functional group, or molecule capable of generating, modifying or modulating a detectable signal. For example, in a radiochemical assay, the labeled binding partner may be labeled with a radioactive isotope of iodine. Alternatively, the labeled binding partner antibody may be labeled with an enzyme, horseradish peroxidase, that can be used in a calorimetric assay. The labeled binding partner may also be labeled with a time-resolved fluorescence reporter or a fluorescence resonance energy transfer (FRET) reporter. Exemplary reporters are disclosed in Hemmila, et al., J. Biochem. Biophys. Methods, vol. 26, pp. 283-290 (1993); Kakabakos, et al., Clin. Chem., vol. 38, pp. 338-342 (1992); Xu, et al., Clin. Chem., pp. 2038-2043 (1992); Hemmila, et al., Scand. J. Clin. Lab. Invest., vol.48, pp. 389-400 (1988); Bioluminescence and Chemiluminescence Proceedings of the 9th International Symposium 1996, J. W. Hastings, et al., Eds., Wiley, New York, 1996; Bioluminescence and Chemiluminescence Instruments and Applications, Knox Van Dyre, Ed., CRC Press, Boca Raton, 1985; I. Hemmila, Applications of Fluorescence in Immunoassays, Chemical Analysis, Volume 117, Wiley, New York, 1991; and Blackburn, et al., Clin. Chem., vol. 37, p. 1534 (1991), the disclosures of which are incorporated herein by reference.

Further examples of labeled binding partners include binding partners that are labeled with a moiety, functional group, or molecule that is useful for generating a signal in an electrochemiluminescent (ECL) assay. The ECL moiety may be any compound that can be induced to repeatedly emit electromagnetic radiation by direct exposure to an electrochemical energy source. Such moieties, functional groups, or molecules are disclosed in U.S. Pat publication 2003-0027357, U.S. Pat. Nos. 5,962,218; 5,945,344; 5,935,779; 5,858,676; 5,846,485; 5,811,236; 5,804,400; 5,798,083; 5,779,976; 5,770,459; 5,746,974; 5,744,367; 5,731,147; 5,720,922; 5,716,781; 5,714,089; 5,705,402; 5,700,427; 5,686,244; 5,679,519; 5,643,713; 5,641,623; 5,632,956; 5,624,637; 5,610,075; 5,597,910; 5,591,581; 5,543,112; 5,466,416; 5,453,356; 5,310,687; 5,296,191; 5,247,243; 5,238,808; 5,221,605; 5,189,549; 5,147,806; 5,093,268; 5,068,088; and 5,061,445; Dong, L. et al., Anal. Biochem., vol. 236, pp. 344-347 (1996); Blohm, et al., Biomedical Products, vol. 21, No. 4: 60 (1996); Jameison, et al., Anal. Chem., vol. 68, pp. 1298-1302 (1996); Kibbey, et al., Nature Biotechnology, vol. 14, no. 3, pp. 259-260 (1996); Yu, et al., Applied and Environmental Microbiology, vol. 62, no. 2, pp. 587-592 (1996); Williams, American Biotechnology, p. 26 (January, 1996); Darsley, et al., Biomedical Products, vol. 21, no. 1, p. 133 (January, 1996); Kobrynski, et al., Clinical and Diagnostic Laboratory Immunology, vol. 3, no. 1, pp. 42-46 (January 1996); Williams, IVD Technology, pp. 28-31 (November, 1995); Deaver, Nature, vol. 377, pp. 758-760 (Oct. 26, 1995); Yu, et al., BioMedical Products, vol. 20, no. 10, p. 20 (October, 1995); Kibbey, et al., BioMedical Products, vol. 20, no. 9, p. 116 (September, 1995); Schutzbank, et al., Journal of Clinical Microbiology, vol. 33, pp. 2036-2041 (August, 1995); Stern, et al., Clinical Biochemistry, vol. 28, pp. 470-472 (August, 1995); Carlowicz, Clinical Laboratory News, vol. 21, no. 8, pp. 1-2 (August 1995); Gatto-Menking, et al., Biosensors & Bioelectronics, vol. 10, pp. 501-507 (July, 1995); Yu, et al., Journal of Bioluminescence and Chemiluminescence, vol. 10, pp. 239-245 (1995); Van Gemen, et al., Journal of Virology Methods, vol. 49, pp. 157-168 (1994); Yang, et al., Bio/Technology, vol. 12, pp. 193-194 (1994); Kenten, et al., Clinical Chemistry, vol. 38, pp. 873-879 (1992); Kenten, Non-radioactive Labeling and Detection of Biomolecules, Kessler, Ed., Springer, Berlin, pp. 175-179 (1992); Gudibande, et al., Journal of Molecular and Cellular Probes, vol. 6, pp. 495-503 (1992); Kenten, et al., Clinical Chemistry, vol. 37, pp. 1626-1632 (1991); Blackburn, et al., Clinical Chemistry, vol. 37, pp. 1534-1539 (1991), Electrogenerated Chemiluminescence, Bard, Editor, Marcel Dekker, (2004), and U.S. Pat. No. 5,935,779, the disclosures of which are incorporated herein by reference. In one embodiment, the electrochemiluminescent group comprises a metal, such as ruthenium or osmium. In one embodiment, the second binding partner is labeled with a ruthenium moiety, such as a tris-bipyridyl-ruthenium group such as ruthenium (II) tris-bipyridine ([Ru(bPy)₃]²⁺).

The term “analyte,” as used herein, means any molecule, or aggregate of molecules, including a cell or a cellular component of a virus, found in a sample. Examples of analytes to which the first binding partner can specifically bind include bacterial toxins, viruses, bacteria, proteins, hormones, DNA, RNA, drugs, antibiotics, nerve toxins, and metabolites thereof. Also included are fragments of any molecule found in a sample. An analyte may be an organic compound, an organometallic compound or an inorganic compound. An analyte may be a nucleic acid (e.g., DNA, RNA, a plasmid, a vector, or an oligonucleotide), a protein (e.g., an antibody, an antigen, a receptor, a receptor ligand, or a peptide), a lipoprotein, a glycoprotein, a ribo- or deoxyribonucleoprotein, a peptide, a polysaccharide, a lipopolysaccharide, a lipid, a fatty acid, a vitamin, an amino acid, a pharmaceutical compound (e.g., tranquilizers, barbiturates, opiates, alcohols, tricyclic antidepressants, benzodiazepines, anti-virals, anti-fungals, antibiotics, steroids, cardiac glycosides, or a metabolite of any of the preceding), a hormone, a growth factor, an enzyme, a coenzyme, an apoenzyme, a hapten, a lectin, a substrate, a cellular metabolite, a cellular component or organelle (e.g., a membrane, a cell wall, a ribosome, a chromosome, a mitochondria, or a cytoskeleton component). Also included in the definition are toxins, pesticide, herbicides, and environmental pollutants. The definition further includes complexes comprising one or more of any of the examples set forth within this definition.

Further examples of analytes include bacterial pathogens such as: Aeromonas hydrophila and other spp.; Bacillus anthracis; Bacillus cereus; Botulinum neurotoxin producing species of Clostridium; Brucella abortus; Brucella melitensis; Brucella suis; Burkholderia mallei (formally Pseudomonas mallei); Burkholderia pseudomallei (formerly Pseudomonas pseudomallei); Campylobacterjejuni; Chlamydia psittaci; Clostridium botulinum; Clostridium botulinum; Clostridium perfringens; Coccidioides immitis; Coccidioides posadasii; Cowdria ruminanitum (Heartwater); Coxiella burnetii; Enterovirulent escherichia coli group (EEC Group) such as Escherichia coli—enterotoxigenic (ETEC), Escherichia coli—enteropathogenic (EPEC), Escherichia coli—O157:H7 enterohemorrhagic (EHEC), and Escherichia coli—enteroinvasive (EIEC); Erlichia spp. such as Erlichia chaffeenis; Franicisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolumi; Mycoplasma mycoides mycoides; Peronoscleropora philippinensis; Phakopsora pachyrhizi; Plesiomonas shigelloides; Ralstonia solanacearum race 3, biovar 2; Rickettsia prowazekii; Rickettsia rickettsii; Salmonella spp; Schlerophthora rayssiae var zeae; Shigella spp.; Staphylococcus aureus; Staphylococcus aureus; Streptococcus; Synchytrium endobioticum; Vibrio cholerae non-O1; Vibrio cholerae O1; Vibrio paraphaemolyticus and other vibrios; Vibrio vulnificus; Xanthormonas oryzae; Xylella fastidiosa (citrus variegated chlorosis strain); Yersinia enterocolitica and Yersinia pseudotuberculosis; and Yersinia pestis.

Further examples of analytes include viruses such as: African horse sickness virus; African swine fever virus; Akabane virus; Avian influenza virus (highly pathogenic); Bhanja virus; Blue tongue virus (Exotic); Camel pox virus; Cercopithecine herpesvirus 1; Chikungunya virus; Classical swine fever virus; Coronavirus (SARS); Crimean-Congo hemorrhagic fever virus; Dengue viruses; Dugbe virus; Ebola viruses; Encephalitic viruses such as Eastern equine encephalitis virus, Japanese encephalitis virus, Murray Valley encephalitis, and Venezuelan equine encephalitis virus; Equine morbillivirus; Flexal virus; Foot and mouth disease virus; Germiston virus; Goat pox virus; Hantaan or other Hanta viruses; Hendra virus; Issyk-kul virus; Koutango virus; Lassa fever virus; Louping ill virus; Lumpy skin disease virus; Lymphocytic choriomeningitis virus; Malignant catarrhal fever virus (Exotic); Marburg virus; Mayaro virus; Menangle virus; Monkeypox virus; Mucambo virus; Newcastle disease virus (VVND); Nipah Virus; Norwalk virus group; Oropouche virus; Orungo virus; Peste Des Petits Ruminants virus; Piry virus; Plum Pox Potyvirus; Poliovirus; Potato virus; Powassan virus; Rift Valley fever virus; Rinderpest virus; Rotavirus; Semliki Forest virus; Sheep pox virus; South American hemorrhagic fever viruses such as Flexal, Guanarito, Junin, Machupo, and Sabia; Spondwendi virus; Swine vesicular disease virus; Tick-borne encephalitis complex (flavi) viruses such as Central European tick-borne encephalitis, Far Eastern tick-borne encephalitis, Russian spring and summer encephalitis, Kyasanur forest disease, and Omsk hemorrhagic fever; Variola major virus (Smallpox virus); Variola minor virus (Alastrim); Vesicular stomatitis virus (Exotic); Wesselbron virus; West Nile virus; Yellow fever virus; and South American hemorrhagic fever viruses such as Junin, Machupo, Sabia, Flexal, and Guanarito.

Further examples of analytes include toxins such as: Abrin; Aflatoxins; Botulinum neurotoxin; Ciguatera toxins; Clostridium perfingens epsilon toxin; Conotoxins; Diacetoxyscirpenol; Diptheria toxin; Grayanotoxin; Mushroom toxins such as amanitins, gyromitrin, and orellanine; Phytohaemagglutinin; Pyrrolizidine alkaloids; Ricin; Saxitoxin; Shellfish toxins (paralytic, diarrheic, neutrotoxic or amnesic) as saxitoxin, akadaic acid, dinophysis toxins, pectenotoxins, yessotoxins, brevetoxins, and domoic acid; Shigatoxins; Shiga-like ribosome inactivating proteins; Snake toxins; Staphylococcal enterotoxins; T-2 toxin; and Tetrodotoxin.

Further examples of analytes include prion proteins such as Bovine spongiform encephalopathy agent.

Further examples of analytes include parasitic protozoa and worms, such as: Acanthamoea and other free-living amoebae; Anisakis sp. and other related worms Ascaris lumbricoides and Trichuris trichiura; Cryptosporidium parvum; Cyclospora cayetanensis; Diphyllobothrium spp.; Entamoeba histolytica; Eustrongylides sp.; Giardia lamblia; Nanophyetus spp.; Shistosoma spp.; Toxoplasma gondii; and Trichinella.

Further examples of analytes include fungi such as: Aspergillus spp.; Blastomyces dermatitidis; Candida; Coccidioides immitis; Coccidiodes posadasil; Cryptococcus neoformans; Histoplasma capsulatum; Maize rust; Rice blast; Rice brown spot disease; Rye blast; Sporothrix schenckii; and wheat fungus.

Further examples of analytes include genetic elements, recombinant nucleic acids, and recombinant organisms, such as:
(1) nucleic acids (synthetic or naturally derived, contiguous or fragmented, in host chromosomes or in expression vectors) that can encode infectious and/or replication competent forms of any of the select agents.
(2) nucleic acids (synthetic or naturally derived) that encode the functional form(s) of any of the toxins listed if the nucleic acids:
   (i) are in a vector or host chromosome;
   (ii) can be expressed in vivo or in vitro; or
   (iii) are in a vector or host chromosome and can be expressed in vivo or in vitro; and
(3) viruses, bacteria, fungi, and toxins that have been genetically modified.

Further examples of analytes include immune response molecules to the above-mentioned analyte examples such as IgA, IgD, IgE, IgG, and IgM.

The term “analog of the analyte,” as used herein, refers to a substance that competes with the analyte of interest for binding to a binding partner. An analog of the analyte may be a known amount of the analyte of interest itself that is added to compete for binding to a specific binding partner with analyte of interest present in a sample. Examples of analogs of the analyte include azidothymidine (AZT), an analog of a nucleotide which binds to HIV reverse transcriptase, puromycin, an analog of the terminal aminoacyl-adenosine part of aminoacyl-tRNA, and methotrexate, an analog of tetrahydrofolate. Other analogs may be derivatives of the analyte of interest.

The term “positive control/calibrator,” as used herein, refers to a known amount of analyte or an analog of the analyte. Positive control/calibrators may be used to assess the proper operation of the instrumentation and/or the sample measurement. Positive control/calibrators may be used as a reference to compare the signal level of the test sample with the signal level of the reference. Positive control/calibrators may also be used along with a mathematical function to relate signal levels with analyte concentrations, one use of which is to convert a signal measurement from a sample to an analyte concentration. The term “positive control/calibrator” encompasses the common definition of both positive control and positive calibrator.

The term “assay positive control/calibrator,” as used herein, refers to reagents used (a) to confirm successful measurement of a sample or (b) to convert a measured signal from a sample into a concentration of the tested analyte. Typically, an assay positive control/calibrator comprises a positive control/calibrator and the reagents used for a binding assay in order to simulate measurements from a sample that contains the analyte.

The invention, in general terms, relate to compositions, kits, and methods that are used as assay positive control/calibrators for binding assays that are used to detect or quantify the amount of an analyte found in a sample. Before use in an assay measurement, the binding partner or partners used in these in assay positive control/calibrators are dry and are co-located in a container with a positive/control calibrator (i.e., a known and dry amount of the analyte or an analog of the analyte).

### Dry Compositions

Certain embodiments of the present invention provide a dry composition comprising:
(a) an optional first binding partner for specifically binding an analyte;
(b) an optional support for binding the first binding partner without blocking the binding of the analyte;
(c) a labeled second binding partner for specifically binding to the same analyte; and
(d) at least one positive control/calibrator reagent comprising a known amount of the analyte or an analog of the analyte that can specifically bind to the second binding partner and, if present, can specifically bind to the first binding partner.

In some embodiments, water or a buffer can be added to the dry composition when it is used in an assay.

In certain embodiments, the sample can be directly combined with the dry composition, e.g., the sample can be added to the dry composition without initially reconstituting the dry composition with water or buffer. In these embodiments, the signal level can be the sum of the analyte signal of the positive control/calibrator reagent and the analyte signal (if any) of the sample.

Various embodiments provide a dry composition comprising:
(a) a labeled binding partner for specifically binding to an analyte; and
(b) at least one positive control/calibrator reagent comprising a known amount of the analyte or an analog of the analyte that can specifically bind to the labeled binding partner.

Certain embodiments provide a dry composition comprising:
(a) a first binding partner for specifically binding an analyte;
(b) a labeled second binding partner for specifically binding to the same analyte; and
(c) at least one positive control/calibrator reagent comprising a known amount of the analyte or an analog of the analyte that can specifically bind to the first and second binding partners.

Some embodiments provide a dry composition comprising:
(a) a first binding partner for specifically binding an analyte;
(b) a support for binding the first binding partner without blocking the binding of the analyte;
(c) a labeled second binding partner for specifically binding to the same analyte; and
(d) at least one positive control/calibrator reagent comprising a known amount of the analyte or an analog of the analyte that can specifically bind to the first and second binding partners.

Various embodiments of the present invention provide a dry composition comprising:
(a) a first binding partner for specifically binding an analyte;
(b) a support that binds to the first binding partner without blocking the binding of the analyte;
(c) a labeled analyte or analog of the analyte that competes with the analyte in a sample for binding to the binding partner; and
(d) at least one positive control/calibrator reagent comprising a known amount of the analyte or an analog of the analyte that can specifically bind to the binding partner.

### Assays

In some embodiments, water or a buffer may be added to the dry composition when it is used in an assay.

This invention can be used with any binding assay technique. See, for example, The Immunoassay Handbook, third edition Wild, Editor, Stockton Press, (2005) and Principles and Practice of Immunoassay, Price and Newman, Editors, Stockton Press, (1997), which are herein incorporated by reference, for descriptions of many such techniques. For convenience, a short description of some binding assay techniques follows.

Binding assay techniques can be subdivided in many ways. For example, some assays require a labeled binding partner for signal detection, while others generate a signal based on the interaction of the analyte and the binding partner—for example, measuring for example, a mass change. Some assays do not use labeled binding partners, but instead use labeled analyte. Some assays use two binding partners to create a sandwich assay, while others use only one binding partner (such as competitive assays). In sandwich assays, both binding partners bind specifically to the same analyte. In some embodiments, the two binding partners bind to differing portions, e.g., differing epitopes, of the analyte. Some assays require a separation step to differentiate between a labeled binding partner that has bound an analyte and a labeled binding partner that has not bound an analyte. Some assays do not require a separation step, such as agglutination assays and assays wherein the label on the labeled binding partner is modified, activated, or deactivated by the binding of the analyte. Some assays require a support in which a binding partner is attached. A support, separation, sandwich assay uses two binding partners—a first binding partner attached to the support, while a second binding partner is a labeled binding partner—to link the label to the support and afterwards washes the support to remove free labeled binding partner before measuring the label.

As used herein, the term “support,” refers to any of the ways for immobilizing binding partners that are known in the art, such as membranes, beads, particles, electrodes, or even the walls or surfaces of a container. The support may comprise any material on which the binding partner is conventionally immobilized, such as nitrocellulose, polystyrene, polypropylene, polyvinyl chloride, EVA, glass, carbon, glassy carbon, carbon black, carbon nanotubes or fibrils, platinum, palladium, gold, silver, silver chloride, iridium, or rhodium. In one embodiment, the support is a bead, such as a polystyrene bead or a magnetizable bead. As used herein, the term “magnetizable bead” encompasses magnetic, paramagnetic, and superparamagnetic beads. In one embodiment, the support is a microcentrifuge tube or at least one well of a multiwell plate.

A binding partner may be immobilized on the support by any conventional means, e.g., adsorption, absorption, noncovalent binding, covalent binding with a crosslinking agent, or covalent linkage resulting from chemical activation of either or both of the support or the first binding partner. In one embodiment, the immobilization of the first binding partner by the support may be accomplished by using a binding pair. For example, one member of the binding pair, e.g., streptavidin or avidin, can be bound to the support and the other member of the same binding pair, e.g., biotin, can be bound to the first binding partner. Suitable means for immobilizing the first binding partner on the support are disclosed, for example, in the Pierce Catalog, Pierce Chemical Company, P.O. Box 117, Rockford, Ill. 61105, 1994, the disclosure of which is incorporated herein by reference for this purpose.

In certain embodiments, the composition can be used as, for example, an assay control for performing a sandwich binding assay. In various embodiments, the analyte or an analog of the analyte is substantially unbound to the first and the second binding partners in the dry composition.

In some embodiments, the support can facilitate the generation or detection of a signal attributable to the sandwich complex formed by binding of the analyte by an immobilized first binding partner and a labeled second binding partner. For example, in an electrochemiluminescent (ECL) assay, the support can be a magnetizable bead. Such magnetizable beads are disclosed in the references listed in the paragraphs defining a labeled binding reagent. In some embodiments, the first binding partner is immobilized on a magnetizable bead and the second binding partner is labeled with a ruthenium moiety, e.g., [Ru(bpy)₃]²⁺, and the generation and detection of an electrochemiluminescent signal is relied upon to identify and/or quantify the presence of the analyte.

In some embodiments, the composition can contain the first and second binding partners in equimolar or equivalent amounts. However, the exact ratio of the first binding partner to the second binding partner may be varied depending on the relative binding specificities of the first and second binding partners, the type of signal relied upon, and other parameters of the assay conditions. Determining the optimum ratio of the first binding partner to the second binding partner for any given set of conditions is within the skill of the average artisan. The desired ratio of first binding partner to the second binding partner may be achieved by simply adding these components to the composition to be dried in that desired ratio.

In certain embodiments, the dry composition can comprise reagents used for a binding assay and a positive control/calibrator reagent. In some embodiments, the composition can be a solid, such as a lyophilized solid.

In certain embodiments, assay positive control/calibrator compositions for performing binding assays can be prepared by:
(a) preparing a first solution comprising a labeled first binding partner and an assay buffer;
(b) freezing the solution formed in (a);
(c) adding a second solution comprising a positive control/calibrator reagent to the frozen mixture at a temperature sufficient to freeze the second solution; and
(d) drying the first and second solutions.

In some embodiments, the first solution further comprises a labeled second binding partner and an optional support that may or may not be pre-associated with the second binding partner.

In various embodiments, assay positive control/calibrator compositions for performing binding assays can be prepared by:
(a) combining a frozen first solution comprising a labeled first binding partner and an assay buffer with a second solution comprising a positive control/calibrator reagent, wherein the combining is performed at a temperature sufficient to freeze the second solution; and
(b) drying the first and second solutions.

In certain embodiments, assay positive control/calibrator compositions for performing binding assays according to the invention can be prepared by:
(a) preparing a first solution comprising a first binding partner for specifically binding an analyte and an assay buffer;
(b) freezing the mixture formed in (a);
(c) adding a second solution to the frozen mixture at a temperature sufficient to freeze the second solution, said second solution comprising a positive control/calibrator reagent and a labeled analyte or analog of the analyte that competes with the analyte in a sample for binding to the binding partner; and
(d) drying the first and second solution.

In some embodiments, assay positive control/calibrator compositions for performing binding assays according to the invention can be prepared by:
(a) combining a frozen first solution comprising a first binding partner for specifically binding an analyte and an assay buffer, with a second solution comprising a positive control/calibrator reagent and a labeled analyte or analog of the analyte that competes with the analyte in a sample for binding to the binding partner, wherein the combining occurs at a temperature sufficient to freeze the second solution; and
(b) drying the first and second solution.

In various embodiments, the first solution further comprises a support that binds to the first binding partner without blocking the binding of the analyte

In certain embodiments, the temperature at which the second solution freezes can be sufficiently low to prevent reaction or binding between reagents from the first solution with reagents from the second solution.

In some embodiments, the combining in (a) can be performed in an assay vessel, for example, a microcentrifuge tube or at least one well of a multiwell plate.

In some embodiments, the dried first and second solutions are in physical contact with one another.

In certain embodiments, the dried first and second solutions can be separated from one another, although they are in the same container. For example, the frozen first solution can be present in a container and combined with the second solution in the same container at a temperature sufficient to freeze the second solution, resulting in frozen first and second solutions that are physically separate from each other. Drying the first and second solutions results in dried first and second solutions separated from one another in the same container.

One skilled in the art will recognize that the components comprising the first solution may themselves be added as one or more separate solutions. Moreover, one skilled in the art will recognize that the invention also encompasses varying the order in which the first and second solutions are added to the assay vessel. For example, the second solution may be added to the assay vessel and frozen before the first solution is added. In some embodiments, the first and second solutions can be frozen separately and combined as frozen solids.

In some embodiments, the first and second solutions can be dried separately, by any means known in the art. Subsequently, the dried first and second solutions can be combined to prepare the inventive composition. Accordingly, certain embodiments provide a method for preparing assay positive control/calibrator compositions for performing binding assays:
(a) preparing a first solution comprising a labeled first binding partner and an assay buffer;
(b) drying the solution formed in (a);
(c) drying a second solution comprising a positive control/calibrator reagent; and
(d) combining the dried first and second solutions.

Some embodiments provide a method for preparing assay positive control/calibrator compositions for performing binding wherein the first solution above further comprises a second binding partner and a support that may or may not be pre-associated with the second binding partner.

Certain embodiments provide a method for preparing assay positive control/calibrator compositions for performing binding assays:
(a) preparing a first solution comprising a first binding partner and an assay buffer;
(b) drying the solution formed in (a);
(c) drying a second solution comprising a positive control/calibrator reagent and a labeled analyte or analog of the analyte that competes with the analyte in a sample for binding to the binding partner; and
(d) combining the dried first and second solutions.

Various embodiments provide a method for preparing assay positive control/calibrator compositions for performing binding wherein the first solution above further comprises a support that may or may not be pre-associated with the binding partner

In certain embodiments, the support can be a bead, for example, a polystyrene or a magnetizable bead.

In some embodiments of the invention, the support can be, for example, the wall of the assay vessel and a first binding partner may be immobilized on the wall of an assay vessel to which a first solution comprising a labeled second binding partner and an assay buffer is added. The first solution can be frozen and a second solution comprising a positive control/calibrator reagent can be added to the frozen first solution at a temperature low enough so that second solution freezes immediately. The frozen first and second solution can then be dried by techniques known in the art. In certain embodiments, the second solution “freezes immediately” if it freezes at a sufficiently fast rate to prevent binding between the binding partners and the positive control/calibrator reagent.

In some embodiments, the dried first and second solutions can be in physical contact with one another. In various embodiments, the dried first and second solutions can be separated from one another, although they are in the same container, i.e., the dried first and second solutions are capable of being in contact with each other depending on, e.g., the orientation of the container.

In certain embodiments, the first and second solutions can be dried by lyophilization. Methods and apparatus for lyophilizing materials, in particular biological materials, are well known to those skilled in the art. Lyophilization has well known uses for material preservation and stability purposes.

In certain embodiments, the compositions of the invention can further comprise a lyophilization buffer. Lyophilization buffers are well known in the art and may contain phosphate buffer and, optionally, one or more cryoprotectants.

The compositions of the present invention can comprise a compound such as trehalose or sucrose. In certain embodiments, both the first and second solutions comprise trehalose or sucrose. In some embodiments, only one of the first and second solutions comprises trehalose or sucrose. In some embodiment, the trehalose or sucrose may exist as a layer between the immobilized capture antibody and the labeled reporter antibody. Such compositions can be formed by adding and freezing a solution comprising trehalose or sucrose after the first solution is frozen, but before the second solution is added.

In certain embodiments, the support can be treated to block or reduce the nonspecific binding of the labeled second binding partner, analyte, or analog of the analyte to the support. Any conventional blocking agents can be used. Suitable blocking agents are described, for example, in U.S. Pat. Nos. 5,807,752; 5,202,267; 5,399,500; 5,102,788; 4,931,385; 5,017,559; 4,818,686; 4,622,293; 4,468,469; and in CA 1,340,320; WO 97/05485; EP-A1-566,205; EP-A2-444,649; and EP-A1-165,669, the disclosures of which are incorporated herein by reference. Exemplary blocking agents include serum and serum albumins, such as animal serum (e.g., goat serum), bovine serum albumin, gelatin, biotin, and milk proteins (“blotto”). The support can be blocked by absorption of the blocking agent either prior to or after immobilization of the first binding partner in the case of sandwich binding assays or of the binding partner in the case of competitive binding assays. In some embodiments, the support can be blocked by absorption of the blocking agent after immobilization of the binding partner. The exact conditions for blocking the support, including the exact amount of blocking agent used, may depend on the identities of the blocking agent and support but may also be determined by using the assays and protocols described in the Examples below.

In some embodiments, the dry composition comprises reagents used for a binding assay and a positive control/calibrator reagent. The dry composition can be placed in many types of containers. In some embodiments, the container can be a multi-well plate that contains, for example, 24, 96, 384,1536, or 6144 wells with each well able to contain one or more dry compositions. In certain embodiments, the multi-well plate, as used in an instrument, can have outside dimensions no larger than about the largest that is specified in the ANSI/SBS 20004 Microplate standards for footprint dimensions (ANSI/SBS 1-2004). The third dimension of the multi-well plate (i.e., the height), as used in an instrument, can have outside dimensions no larger than about 44 mm. In various embodiments, the container can be a tube that is less than or equal to about 9 mm in diameter, and less than or equal to about 40 mm tall. In some embodiments, the container can be a tube that has a maximum outside diameter of about 8.6 mm and a height of about 33.8 mm. In some embodiments, a two-dimensional array of containers can be placed in a holder that is within the multi-well plate dimensions above. In various embodiments, the two-dimensional array of container in the holder can be about 35 mm tall.

In some embodiments, the containers can be hermetically sealed. In some embodiments, the container can be sealed with an elastomeric, thermoset, or a thermoplastic material, such as EVA or Santoprene®, that has been pressed into the container's opening. In some embodiments, the container can be sealed with a laminate comprising a metallic layer, such as a foil microplate seal. In various embodiments, the container can be sealed with a laminate comprising a thermally modifiable layer, such as a laminate that can be heat-sealed to the container. In some embodiments, the container can be sealed with a laminate comprising an adhesive layer that can bond the laminate to the container.

In some embodiments, the container comprises at least one enclosure, such as one or more sealed enclosures (containers) inside a sealed bag. In some embodiments, the sealed bag can, for example, comprise polyethylene, polyester, aluminum, nickel, a trilaminate of polyester-foil-polyethylene, or a bilaminate of polyester-polyethylene. In some embodiments, a desiccant can be added between the innermost enclosure and the outermost enclosure. The desiccant can, for example, comprise calcium oxide, calcium chloride, calcium sulfate, silica, amorphous silicate, aluminosilicates, clay, activated alumina, zeolite, or molecular sieves. In some embodiments, a humidity indicator can be added between the innermost enclosure and the outermost enclosure. The humidity indicator can, for example, be used as an indication that the dry composition is still sufficiently dry that its stability has not been compromised. In some embodiments, the humidity indicator can be viewed through the outermost enclosure. In certain embodiments, the humidity indicator can be a card or disc wherein the humidity is indicated by a color change, such as one designed to meet the US military standard MS20003.

In some embodiments, the humidity barrier created by the container can be sufficient to keep the dry composition dry when the external conditions are 45° C. and 100% relative humidity for 10 days, 20 days, 40 days, 67 days, 3 months, 6 months, 12 months, 18 months, 24 months, or longer.

In some embodiments, the humidity barrier created by the container can be sufficient to keep the dry composition dry when the external conditions are 25° C. and 100% relative humidity for 1 day, 1 week, 1 month, 3 months, 6 months, 12 months, 18 months, 24 months, or longer.

In some embodiments, the humidity barrier created by the container can be sufficient to keep the dry composition dry when the external conditions are 4° C. and 100% relative humidity for 3 months, 6 months, 12 months, 18 months, 24 months, or longer.

Certain embodiments of the invention provide a method for detecting and/or quantifying an analyte utilizing a non-competitive assay, comprising:
(a) providing a sample, which may contain the analyte;
(b) forming a test reaction mixture by combining the sample with a composition comprising:
   (i) an optional first binding partner for specifically binding an analyte;
   (ii) an optional support for binding the first binding partner without blocking the binding of the analyte; and
   (iii) a labeled second binding partner for specifically binding to the same analyte;
(c) forming at least one positive control/calibrator reaction mixture by combining the sample with at least one dry composition comprising:
   (i) said optional first binding partner;
   (ii) said optional support;
   (iii) said labeled second binding partner; and
   (iv) at least one positive control/calibrator reagent comprising a known amount of the analyte or an analog of the analyte that can specifically bind to the second binding partner and, if present, can specifically bind to the first binding partner;
(d) incubating the test reaction mixture and the at least one positive control/calibrator reaction mixture; and
(e) measuring a signal attributable to a complex formed by binding the analyte to the labeled binding partner for each of the reaction mixtures.

In some embodiments, the method for detecting and/or quantifying an analyte further comprises:
(f) assessing the data, e.g., the measured signal from the at least one positive control/calibrator reaction mixtures by performing at least one step selected from:
   (i) confirming successful measurement of the sample; and
   (ii) converting the signal generated from the test reaction mixture into a concentration of the test analyte.

In certain embodiments, the method can be a sandwich binding assay method.

Various embodiments of the invention provide a method for detecting and/or quantifying an analyte utilizing a competitive binding assay, comprising:
(a) providing a sample, which may contain the analyte;
(b) forming a test reaction mixture by combining the sample with a composition comprising:
   (i) a binding partner for specifically binding the analyte;
   (ii) an optional support that binds to the binding partner without blocking the binding of the analyte; and
   (iii) a labeled analyte or analog of the analyte that competes with the analyte in the sample for binding to the binding partner;
(c) forming at least one positive control/calibrator reaction mixture by combining the sample with at least one dry composition comprising:
   (i) said binding partner;
   (ii) said optional support;
   (iii) said labeled analyte or analog of the analyte; and
   (iv) at least one positive control/calibrator reagent comprising a known amount of the analyte or an analog of the analyte that can specifically bind to the binding partner;
(d) incubating the test reaction mixture and the at least one positive control/calibrator reaction mixture; and
(e) measuring a signal attributable to a complex formed by binding the analyte to said binding partner for each of the reaction mixtures.

In some embodiments, the method for detecting and/or quantifying an analyte further comprises:
(f) assessing the data, e.g., the measured signal, from the at least one positive control/calibrator reaction mixtures by performing at least one step selected from:
   (i) confirming successful measurement of the sample; and
   (ii) converting the signal generated from the test reaction mixture into a concentration of the test analyte.

The following paragraph describes additional embodiments of both the non-competitive and the competitive assays above. In some embodiments, the composition in (b) can be a dry composition, such as any of the dry compositions described herein. In certain embodiments, water or a buffer can be added to the dry composition(s) when it is used in an assay. In some embodiments, the dry composition(s) can be directly combined with the sample, e.g., without first reconstituting the dry composition(s) with water or buffer. In certain embodiments, the support can be a bead, for example, a polystyrene or a magnetizable bead. In some embodiments, the second binding partner can be labeled with an ECL moiety. In a various embodiments, the ECL moiety can be [Ru(bpy)₃]²⁺. In certain embodiments, “detecting an analyte” refers to determining the presence of an analyte. That is, the sample suspected of containing the analyte may or may not contain the analyte. In some embodiments, “quantifying an analyte” refers to determining the amount of analyte present in the sample. The sample may be known to contain the analyte but in unknown amount. Alternatively, the sample may or may not contain the analyte and the method comprises both detecting and quantifying the analyte.

The following paragraph describes additional embodiments of both the non-competitive and the competitive assays above. In some embodiments, the data from the at least one positive control/calibrator reaction mixtures can be used to confirm successful measurement of the sample. The measured signal from these mixtures can be compared to a pre-determined signal range. If the measured signal is within the pre-determined signal range, the measurement may be deemed valid and measurements from the test reagent mixture reported. If the measured signal is not within the pre-determined signal range, re-calibration may be necessary. The measured signal from the positive control/calibrator reaction mixture can also be converted to an analyte concentration through the use of a mathematical calibration curve. This converted value can be compared to a pre-determined concentration range; if the measured concentration is within the pre-determined range, the measurement may be deemed valid and measurements from the test reagent mixture reported.

In various embodiments in which the sample is added directly to a positive control/calibrator reaction mixture (i.e., to reconstitute the mixture), the concentration of analyte in the sample can be determined from the measured signal by an algorithm that can attribute a portion of the measured signal to the sample, e.g. a mathematical function to relate signal levels with analyte concentrations. For example, the signal measured from a positive control/calibrator reaction mixture reconstituted with water or a buffer solution can be compared to the signal measured from the positive control/calibrator reaction mixture reconstituted with the sample. In such embodiments, the difference between the two signals, if any, can be used to calculate the amount of analyte in the sample and the control/calibrator acts as an internal control.

In the embodiments that use more than one positive control/calibrator reaction mixtures (e.g., two or three), the analyte concentrations may differ or may be the same. All or only some of the plurality of positive control/calibrator reaction mixtures can be used to determine whether or not a measurement from the test reagent mixture is reported. For example, the Bio Rad Immunoassay Plus Controls level 2 and 3 from Example 7, infra, can be used in the positive control/calibrator reagents to confirm successful measurement of the sample.

The following paragraph describes additional embodiments of both the non-competitive and the competitive assays above. In some embodiments, the data from the at least one positive control/calibrator reaction mixtures can be used to convert the signal generated from the test reaction mixture into a concentration of the test analyte by helping create a mathematical calibration curve. Calibration curves enable both interpolation and extrapolation of signal measurements for samples with known analyte concentrations for signal measurements of samples unknown amounts of analyte. The form of the mathematical functions used in the curve fit may make assumptions of continuity and/or smoothness of the underlying relation by interpolating the measurements with function such as piecewise constant, piecewise linear, cubic spline, or by fitting all the data with a linear, quadratic, cubic, or quartic polynomials while for overconstrained systems parameters are computed by minimizing an error function such as least squares (e.g., Press, W., Teukolsky, S. Vetterling W., Flannery, B. Numerical Recipes in C The Art of Scientific Computing. Second Edition. 1992. Cambridge University Press.) or total least squares (e.g., Van Huffel, S. and Vandewalle, J. The Total Least Squares Problem Computational Aspects and Analysis. 1991. Society for Industrial and Applied Mathematics). The form of the mathematical function may make assumptions about the assay mechanism, such as a one site saturation, two site saturation, one site saturation with nonspecific binding, two site saturations with nonspecific binding, a sigmoidal dose response curve with or without a variable slope, one-site competition, two-site competition, or a four-parameter logistic. Generation of the calibration curve can entail selecting the form of the mathematical function and then fitting the parameters of the function with measurements. The measurements can be done on the instrument or can be done in part or wholly elsewhere (e.g., at the place the assay is manufactured). The measurements can perfectly constrain or over-constrain the mathematical function. For overconstrained systems, model parameters can be computed by minimizing an error function such as least squares (e.g., Press et al. 1992) or total least squares (e.g., Van Huffel et al. 1991). For example, the PSA calibrators A through G from Example 7, infra, can be used with a four-parameter logistic function to construct a mathematical calibration curve used to convert the signal generated from the test reaction mixture into a concentration of the test analyte.

In certain embodiments, the binding assay methods of the invention comprise incubating the sample with the composition prior to the measuring step. The incubation time can be on the order of minutes, such as a time of less than 60 minutes, or a time ranging from 1 to 30 minutes. The incubation can be performed at a temperature ranging from greater than about 0° C. to about 50° C., such as about room temperature or about 37° C. Other temperatures are achievable by means of a heating or cooling bath or other temperature adjustment means known to the art. The incubation can be carried out with stirring or with agitation by means of, for example, a stirrer or shaker.

In certain embodiments, the assay can be a single-step assay, where the assay reagents and, where used, the at least one positive control/calibrator can be contained in one composition to which an appropriately diluted sample can be added. In some embodiments, the assay can be a two-step assay, where the assay reagents and, where used, the at least one positive control/calibrator can be contained in one composition to which a sample and an appropriate diluent can be added. “Single-step” and “two-step” as used herein refer to the process required to actually perform the analyte binding event. Single- and two-step assays can incorporate other processes subsequent to the analyte binding event, such as the preparation of the sample for measuring. For example, in an ECL assay, an assay buffer containing piperazine-1,4-bis(2-ethanesulfonic acid) (PIPES); tri-n-propylamine; N,N,N′,N′-Tetrapropyl-1,3-diaminopropane; and/or salts thereof can be added to the assay mixture to facilitate the ECL measurement as described, for example, in U.S. Pat. No. 6,451,225, which is incorporated by reference herein. For an additional example, a single- or two-step assay can also comprise the transfer of the reaction mixture to a measuring cell, for example an electrochemical cell as described in U.S. Pat. No. 6,325,973, which is incorporated by reference herein. A single step method can simplify the assay process eliminating the sequential addition and transfer of multiple reagents to perform the assay, such as the assay reagents and positive control/calibrators.

In certain embodiments, the methods of the invention comprise assaying multiple analytes (i.e, two or more analytes) in a single sample. Multiple calibrators can be used per analyte measured, potentially forming an array of calibrators for the particular analyte type where each analyte type will be assessed with positive control/calibrator reagents of varying analyte concentration. The assay can be used in conjunction with a multi-well tray, such as a 96-well tray or other multi-well trays known in the art.

The exact steps and means of measuring or detecting the signal attributable to the complex formed by the binding of the immobilized capture antibody and the labeled reporter antibody to the analyte can depend on the exact nature of the labeled binding partner, analyte, or analog of the analog and also on the support to which the binding partner is immobilized. Such techniques are well known in the art. For example, if the labeled component of the assay mixture is labeled with a radioactive atom, then the signal can be detected by means of a scintillation counter. Alternatively, if the labeled component of the assay mixture is labeled with an ECL moiety, a chemiluminescent moiety, or a fluorescent moiety, then the signal can be detected using a light detector such as a CCD, a photomultiplier tube, a photodiode, a CMOS detector, an NMOS detector, a phototransistor or an avalanche photodiode.

After the signal from the labeled assay component is measured, the presence and/or amount of the analyte can be determined by comparing a property of the detected signal, e.g., intensity, amplitude, duration, etc., to a known or previously measured correlation between that property and the presence or the amount of the analyte. Such methods are well known to those skilled in the art.

The sample, which may contain the analyte, can be drawn from any source which it is desired to analyze. For example, the sample can arise from body or other biological fluid, such as blood, plasma, serum, milk, semen, amniotic fluid, cerebral spinal fluid, sputum or saliva. Alternatively, the sample can be a water sample obtained from a body of water, such as lake or river. The sample can also prepared by dissolving or suspending a sample in a liquid, such as water or an aqueous buffer. The sample source can be from air; for example, the air can be filtered; the filter washed by a liquid; thereby transferring an analyte from the air into the liquid. The sample can be subjected to a treatment or processing, such as filtration or pH adjustment, prior to the assay procedure. The sample can further comprise or have added to it an agent that facilitates the generation or detection of the signal attributable to the complex formed by binding of the immobilized capture antibody and the labeled reporter antibody to the analyte. For example, when the reporter antibody is labeled with an enzyme, the sample can further comprise or have added to it a substrate for that enzyme.

### Kits

In some embodiments, the present invention also provides kits comprising
(a) at least one dry composition comprising reagents used for a binding assay and a positive control/calibrator reagent;
(b) at least one container in which each of the at least one dry compositions are located;
(c) calibration/control information or a key for obtaining calibration/control information.
(d) optional written instructions in the form of an insert or packaging which describe how to use the present kit; and

In certain embodiments, the dry composition can be any of the dry compositions described herein. The dry composition can comprise at least one reagent used for a binding assay selected from:

a labeled binding partner for specifically binding an analyte

a second binding partner for specifically binding to the same analyte where the labeled binding partner is a first binding partner; and

at least one support that can bind or is bound to the first binding partner.

In certain kits according to the invention, the reagents comprise a label that is an ECL moiety. In related embodiments, the label can be a ruthenium or osmium-containing ECL moiety. In yet other related embodiments of the invention, the ECL moiety can be [Ru(bpy)₃]²⁺.

In some embodiments of the invention, the reagents comprise a bead. In related embodiments, the bead can be a magnetizable bead.

The kit compositions may comprise any of the reagents described herein as well as any of the containers, humidity indicators, and humidity barriers described herein.

In some embodiments, calibration/control information included in the kit can be the valid signal range or the valid concentration range for the positive control/calibrators. In some embodiments, the form of the mathematical calibration curve can be included. In some embodiments, the form of the mathematical calibration curve and some or all of the curve's parameters can be included. In some embodiments, calibration/control information can be contained in the kit only via an identifying key that is used to look up the calibration/control information stored elsewhere. The identifying key can be, for example, as simple as the name of the analyte to be tested, and the information could be stored, for example, in the operator's manual for the instrument or assay kit or in software for the instrument or assay kit. The identifying key can comprise, for example, an numerical string, an alphanumeric string, or a binary string. The identifying key can, for example, be bar-coded on the assay kit to ease entry into an instrument wherein the calibration/control information is stored.

In some of these embodiments, a negative control/calibrator may be used. By having a negative control/calibrator and a positive control/calibrator, interpolation can be used to determine analyte concentration. By having a negative control/calibrator a tighter threshold on the presence or absence of the analyte in a sample may be used. Samples lacking analyte typically nevertheless produce a measurable signal, called a background signal. The background signal has many possible sources. For example, nonspecific binding of the labeled binding partner can cause a background signal. Some background signal sources can be detection method specific, for example, background radiation for isotope detection and auto-fluorescence for fluorescent measurements. Some sources of the background signal can be sample-specific. The environment may also affect the signal, through for example, temperature, pressure and/or other dependences. Because the sample may contain the analyte, using the sample to rehydrate the negative control/calibrator may add extra steps in the analyte determination. Because the sample may contain compounds that either enhance or reduce the specific signal for a given analyte concentration, using a buffer rather than the sample to rehydrate the negative control/calibrator may also add extra steps in the analyte determination. Removal of the labeled binding partner from the reaction mixture may reduce the signal modulation due to non-specific binding of the labeled binding partner. In some embodiments, a third binding partner can be used in lieu of the first binding partner in embodiments that use a support. The third binding partner can (1) not specifically bind the analyte, and/or (2) have similar non-specific binding properties as the first binding partner; for example, they can both be antibodies or fragments thereof. In embodiments comprising a support, the third and first binding partners can both bind the support. Using the sample to rehydrate a negative control with the third binding partner in lieu of the first binding partner can generate signal levels comparable to a sample lacking the analyte—with similar matrix effects, nonspecific binding, and other assay effects.

Another embodiment provides a kit comprising at least one dry composition for use as an assay positive control/calibrator comprising:

(a) a labeled binding partner comprising a label and a binding partner wherein said labeled binding partner can specifically bind to an analyte; and

(b) a positive control/calibrator reagent comprising a known amount of the analyte or an analog of the analyte that can specifically bind to the binding partner;

wherein the composition has a moisture content of less than or equal to about 5% by weight, relative to the total weight of the composition, and

wherein each of the at least one dry composition is positioned within a container.

In one embodiment, the labeled binding partner and positive control/calibrator reagent are in physical contact with each other, e.g., positioned within the container such that they are capable of contacting each other, e.g., depending on, e.g., the orientation of the container. In one embodiment, the dry composition comprises an intimate physical mixture. In another embodiment, “physical contact” comprises at least two adjoining regions in physical contact, wherein at least one first region comprises the labeled binding partner and at least one second region comprises the positive control/calibrator reagent. Any container can be used as known in the art, e.g., tubes, bottles, or vessels such as those described in, e.g., FIG. 16 of U.S. Provisional Application No. 60/693,041, “Portable Diagnostic Testing Instrument,” filed Jun. 23, 2005, the disclosure of which is incorporated herein by reference.

In some embodiments, the kit comprises positive control/calibrator reagents for that allow the analyst to span the range (or a portion of the range) of measurable or detectable concentrations. In one embodiment, the analyte has a measurable concentration ranging from c1 to c2, wherein c1<c2. The kit can further comprise:

(a) a first dry composition comprising reagents used for a binding assay and a positive control/calibrator reagent having p distinct, known amounts of the analyte or an analog of the analyte, p≧1;

(b) a second dry composition comprising reagents used for a binding assay without the positive control/calibrator reagent;

(c) at least one container in which the compositions are located;

(d) calibration/control information, or a key for obtaining calibration/control information;

wherein when rehydrated by a reagent lacking the analyte, the p known amounts create calibration concentrations of d₁, d₂, . . . , dₚ and wherein d₁<d₂ . . . <dₚ;

wherein the maximum of (i) d₁/c1, (ii) dₘ₊₁/dₘ for 1≦m≦p−1, and (iii) c2/dₚ is less than or equal to about α;

wherein α=2 (c2/((p−1)×c1)) if p>1 and 2 (c2/c1) if p=1.

In one embodiment, α=2 (c2/(p×c1)). In another embodiment, α=2 (c2/(p+1)c1)). In yet another embodiment, p=1, 2, 3, or 4.

### EXAMPLES

These Examples describe a quantitative electrochemiluminescent (ECL) based sandwich immunoassay for the detection of prostate specific antigen (PSA) utilizing a lyophilized composition comprising an immobilized first antibody, a labeled second antibody, and a known amount of PSA.

In these Examples, reagents for detecting prostate antigen were lyophilized and tested for their ability to detect PSA in three matrices. Results showed that assay performance using lyophilized reagents compared favorably to assay performance using wet reagents.

Initially, a brief optimization for PSA reagents was performed specifically for the ORIGEN® Analyzer (BioVeris Corp.). This was followed by a determination of the incubation time required for assay equilibrium. The high affinity, high avidity monoclonal antibodies used in this assay allow for rapid (30 minutes or less) quantitative detection of PSA. In this Example, wet reagents were directly compared to lyophilized reagents, as well as other ECL based systems including the Elecsys 1010 (Roche Diagnostics Corp.), and the M-SERIES M1 Analyzer (BioVeris Corp.), which presently use wet reagents. In all systems, PSA concentrations derived from assay specific standard curves were similar. Assay performance was similar at clinically relevant levels for this analyte. In order to directly compare reagents, the same reagents were tested on the Elecsys 1010, ORIGEN and M1 analyzers. Matrices tested in this Example included spiked calibrator diluent, whole blood, and plasma. Detection in calibrator diluent was similar in all systems tested, as was detection in plasma.

### Example 1

### Labeling of Antibodies

Monoclonal antibody PSA10 (304-01, CanAg Diagnostics) was biotinylated by incubating at room temperature for 1 h on a rotating mixer with Biotin NHS-ester LC (11015, BioVeris Corp.) added at a 2-fold molar excess. Unreacted Biotin-NHS-ester LC was removed by gel filtration on a Sephadex® G-25 column swelled in phosphate-buffered saline (PBS)/0.05% sodium azide. Dialysis (Slide-A-Lyzer® Dialysis Cassettes, Pierce) involved two room temperature dialysis exchanges (3 h each) and an overnight dialysis against PBS/0.05% sodium azide at 4° C. Protein concentration was determined by bicinchoninic acid (BCA) protein assay. The biotinylated first antibodies were stored at 4° C.

Monoclonal antibody PSA66 (310-01, CanAg Diagnostics) was ruthenylated with a 7.5-fold molar excess of ORI-TAG® NHS ester. Unreacted ORI-TAG® was removed by the same procedure described above for the purification of the biotinylated antibody. ORI-TAG®:protein incorporation ratios were determined by dividing the molarity of the ORI-TAG® by the molarity of the ORI-TAG® labeled protein. ORI-TAG® labeled second antibodies (detection reagent) were stored at 4° C.

### Example 2

### Preparation of analyte-specific Beads

Biotinylated first antibodies were immobilized on 2.8 μm streptavidin-coated superparamagnetic beads. The streptavidin beads were prewashed twice with double the original bead suspension volume of PBS, 0.3% Tween® 20 (PBS-T) using a magnetic microparticle separator (Dynal) to capture the beads before buffer removals. The streptavidin beads were then washed once more with PBS without Tween® 20. Streptavidin beads were reconstituted to their original volume with PBS without Tween® 20. The biotinylated antibodies (100 μg) were incubated with 1 mL pre-washed streptavidin beads for 1 h at room temperature on a rotator to keep the beads in suspension. The prewash procedure was repeated after the incubation period to remove any free biotinylated antibody. The first antibody-coated superparamagnetic beads (capture beads) were stored at 4° C.

### Example 3

### Assay Protocols

For the ECL measurements, an ORIGEN® I analyzer (BioVeris Corp.), M-SERIES® M1 Analyzer (BioVeris), and Elecsys® 1010 (Roche-Diagnostics-USA) were used. The ORIGEN® integrates a luminometer, potentiostat, electrochemical flow cell, fluid-handling components, and a 50-tube carousel. The instrument is controlled by a microcomputer via operator manipulation of on-screen menus.

A reagent optimization was performed for assays on the ORIGEN® and M1 analyzers. The sample to be assayed (50 μL) was added to 12×75 mm reaction tubes or microtiter plate wells. Also added were capture beads (25 μL) diluted 1:50 in a bead diluent containing sucrose, and 25 μL detection reagent diluted to 5 μg/mL in an antibody diluent. These concentrations refer to the working concentration of reagents added to the reaction tube well and not to the final concentration in the reaction tube well. PBS (200 μL) and 0.5% Triton X 100 (assay diluent) were added to bring the final volume to 300 μL. Reactions were allowed to incubate for 30 min on the carousel of the ORIGEN® or on a plate shaker (except for the incubation time study where incubation times were varied). When lyophilized reagents were used on the ORIGEN®, the sample was added directly to the reaction tube containing the lyophilized reagent pellet followed by addition of 250 μL assay diluent. Lyophilized PSA calibrators received 300 μL assay diluent.

Assays on the Elecsys® 1010 were run using customer and WDPT (research) software. Unlike the customer software, the research software provides the user with ECL values allowing the user to perform their own linear regression analysis. Elecsys® total PSA (Elecsys® tPSA) was tested with the commercially available tests using customer software according to the manufacturer's protocol. ORIGEN® Demonstration PSA Reagents (Elecsys® PSA Demo) were run on the Elecsys® 1010 with research software. Self-prepared rack packs were also prepared with the same reagents and diluents that were used on the ORIGEN® and M1 Analyzers and run with a similar protocol. This made a direct comparison of platforms possible.

### Example 4

### Whole Blood/Plasma Sampling

Whole blood samples containing a lithium heparin anticoagulant were received from Research Sample Bank and spiked to 4 levels (including a negative) with PSA and designated as 1, 2, 3, and 4. Plasma was prepared from each whole blood spike level. Whole blood and plasma spikes were assayed according to the procedures described above. With each run, a calibrator set, calibrator diluent (CD) spikes, whole blood and/or plasma spikes, and Bio Rad controls were assayed.

### Example 5

### Lyophilization of Reagents

Working solutions of capture and detection reagents were prepared as described above and combined in equal parts to produce a bulk formulation of assay reagent.

To prepare compositions containing the bulk assay reagent and positive controls, the assay reagents and positive controls were added separately to tubes on dry ice to prevent antibody/antigen binding in the tubes. The bulk assay reagent was first added to polypropylene tubes in a tray that was on a bed of dry ice. The reagent froze immediately upon addition to the tube. Next, 50 μL of PSA antigen containing calibrator (A-G) was added on top of the frozen reagent pellet, causing it to freeze immediately. Two distinct frozen pellets were observed. These were lyophilized using the same protocol used for the reagent only containing tubes. Once the lyophilization cycle was complete, all tubes were back-filled under argon, stoppered, and crimp sealed (“Lyophilized PSA calibrators”).

Fifty microliters of this bulk assay reagent were added to polypropylene tubes and lyophilized over 16.5 h in accordance with the drying phase parameters provided below.

Drying Phase Parameters

| Step | Temp (C.) | Time (min) | Pressure (mT) | Ramp/Hold |
|---|---|---|---|---|
| 1 | −18 | 30 | 10 | Hold |
| 2 | −8 | 180 | 10 | Ramp |
| 3 | −8 | 240 | 10 | Hold |
| 4 | 0 | 180 | 10 | Ramp |
| 5 | 0 | 180 | 10 | Hold |
| 6 | +20 | 180 | 10 | Ramp |

The term “hold” in the table refers to holding the temperature and pressure to the stated temperature and pressure for the stated time on that line. The term “ramp” in the table means to start at the previous step's conditions and change them over the stated time to the stated temperature and pressure on that line.

### Example 6

### Incubation Times

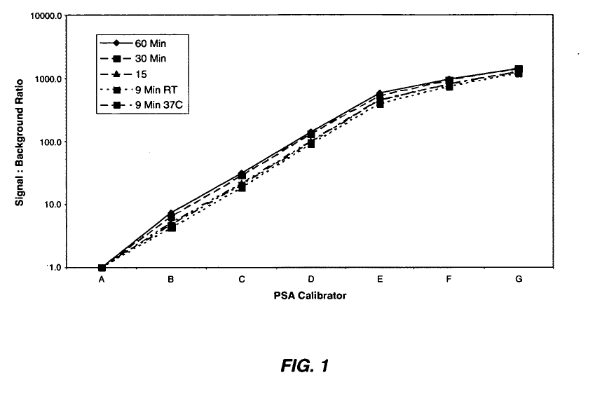

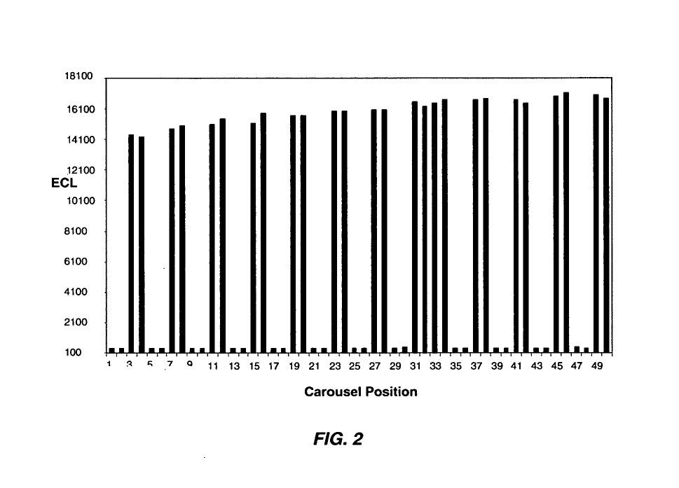

Samples for assay on the ORIGEN® were incubated for 9, 15, 30, or 60 minutes at room temperature or at 37° C. for 9 minutes. On the ORIGEN®, assay equilibrium appeared to be reached after approximately 30 min of incubation at room temperature. There was no significant difference in dose response curves generated after a 9 min incubation at 37® C. and room temperature. FIG. 1 shows dose-response curves generated at various time points. FIG. 2 shows alternating pairs of negative and positive reactions after a 30 min incubation.

### Example 7

### Comparison of Platforms

PSA-containing samples were assayed on the Elecsys®, ORIGEN®, and M1 analyzers. The PSA calibrators prepared from BioVeris PSA Demonstration Reagents were tested along with CD, whole blood, and plasma spikes. Bio Rad Immunoassay Plus Controls levels 1, 2, and 3 were run with each assay to check the precision of each assay. Bio Rad 2 and 3 have stated concentration ranges of 3.0-4.9 ng/mL and 17-28 ng/mL, respectively.

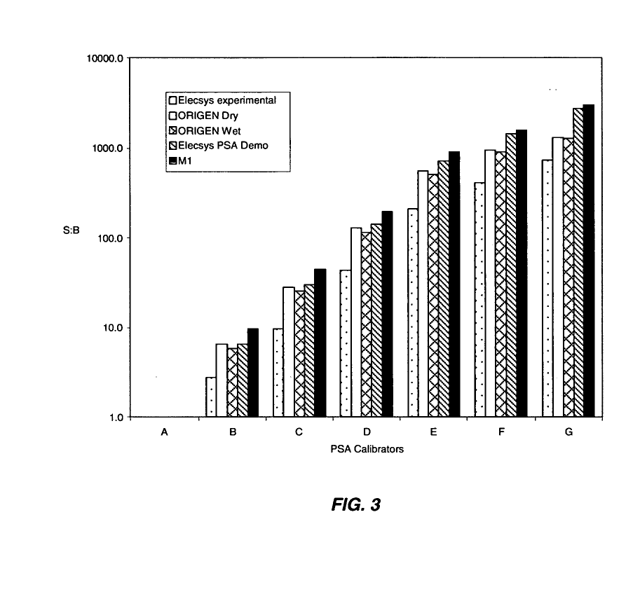

FIG. 3 is a graphical representation of S:B (signal:background) ratios for calibrator sets for all instruments (ORIGEN®, Elecsys®, and M1) The data are shown in Table I, below. In all assays, linear dose response curves with high correlation coefficients were generated. There was no significant difference in performance between lyophilized and wet reagents on the ORIGEN®.

TABLE I

| | | | | | | | | | | PSA | |
|---|---|---|---|---|---|---|---|---|---|---|---|
| | | Elecsys PSA | | | | | | | | Experimental | |
| | Elecsys | Demo | | ORIGEN Wet | | ORIGEN Dry | | M1 | | (Elecsys) | |
| | tPSA | Mean | | Mean | | Mean | | Mean | | Mean | |
| Sample | [ng/ml] | ECL | S:B | ECL | S:B | ECL | S:B | ECL | S:B | ECL | S:B |
| Cal A | 0.008 | 623 | 1.0 | 410 | 1.0 | 414 | 1.0 | 328 | 1.0 | 4112 | 1.0 |
| Cal B | 0.3605 | 4127 | 6.6 | 2388 | 5.8 | 2746 | 6.6 | 3213 | 9.8 | 11522 | 2.8 |
| Cal C | 1.79 | 18678 | 30.0 | 10582 | 25.8 | 11641 | 28.1 | 14576 | 44.4 | 40429 | 9.8 |
| Cal D | 8.435 | 87592 | 140.7 | 46984 | 114.7 | 53320 | 128.8 | 64748 | 197.4 | 180958 | 44.0 |
| Cal E | 41.36 | 443195 | 712.0 | 209942 | 512.7 | 229103 | 553.4 | 293716 | 895.5 | 861689 | 209.6 |
| Cal F | 88.65 | 882033 | 1416.9 | 367497 | 8974 | 387227 | 935.3 | 513966 | 1567.0 | 1702011 | 414.0 |
| Cal G | 176.77 | 1711396 | 2749.2 | 528676 | 1291.0 | 543969 | 1313.9 | 992313 | 3025.3 | 3023451 | 735.4 |
| CD 1 | 0.007 | 588 | 1.0 | 408 | 1.0 | 454 | 1.0 | 404 | 1.0 | 4225 | 1.0 |
| CD 2 | 1.525 | 16424 | 28.0 | 10405 | 25.5 | 11193 | 24.7 | 13588 | 33.6 | 36494 | 8.6 |
| CD 3 | 5.68 | 59186 | 100.7 | 36181 | 88.8 | 37801 | 83.3 | 46522 | 115.2 | 123566 | 29.2 |
| CD 4 | 26.96 | 276349 | 470.4 | 154431 | 379.0 | 159044 | 350.3 | 197434 | 488.7 | 564832 | 133.7 |
| Plasma | <0.006 | 510 | 1.0 | 420 | 1.0 | 419 | 1.0 | 273 | 1.0 | 5493 | 1.0 |
| 1 | | | | | | | | | | | |
| Plasma | 1.475 | 14051 | 27.6 | 8250 | 19.6 | 8895 | 21.3 | 10432 | 38.3 | 33841 | 6.2 |
| 2 | | | | | | | | | | | |
| Plasma | 5.395 | 52239 | 102.5 | 28288 | 67.4 | 30572 | 73.1 | 35621 | 130.7 | 108966 | 19.8 |
| 3 | | | | | | | | | | | |
| Plasma | 26.675 | 244266 | 479.4 | 130729 | 311.3 | 143443 | 342.8 | 173144 | 6354 | 516315 | 940 |
| 4 | | | | | | | | | | | |
| WB1 | ND | ND | ND | 525 | 1.0 | 472 | 1.0 | 280 | 1.0 | ND | ND |
| WB2 | ND | ND | ND | 3281 | 7.3 | 3174 | 6.7 | 3910 | 14.0 | ND | ND |
| WB3 | ND | ND | ND | 13048 | 24.9 | 12817 | 27.2 | 9426 | 33.7 | ND | ND |
| WB4 | ND | ND | ND | 54220 | 1034 | 59459 | 126.0 | 43422 | 155.4 | ND | ND |
| BioRad | 0.631 | 6499 | 10.4 | 3771 | 9.2 | 3943 | 9.5 | 3273 | 10.0 | 15086 | 3.7 |
| 1 | | | | | | | | | | | |
| BioRad | 3.61 | 34805 | 55.9 | 19873 | 48.5 | 20357 | 49.2 | 17810 | 54.3 | 67653 | 16.5 |
| 2 | | | | | | | | | | | |
| BioRad | 20.32 | 196315 | 315.4 | 113605 | 277.4 | 114153 | 275.7 | 102823 | 313.5 | 368077 | 89.5 |
| 3 | | | | | | | | | | | |

Cal—Calibrator
CD—Calibrator Diluent
WB—Whole Blood
ND—Not Determined

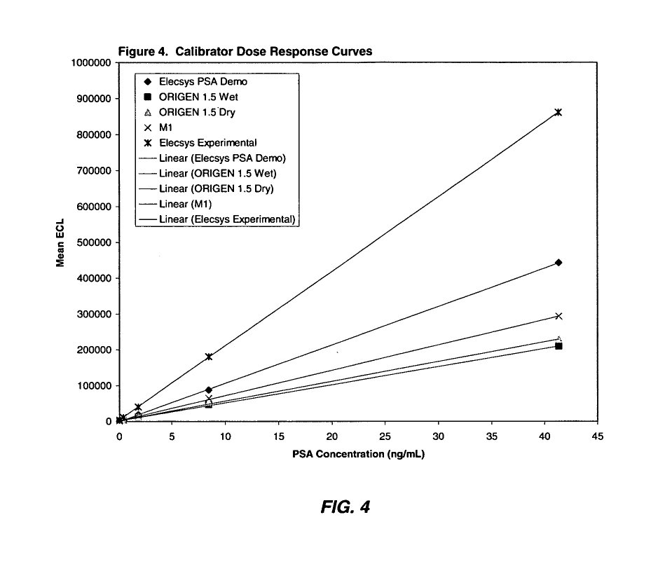

Concentrations of spikes were derived from calibration curves generated for each instrument with PSA calibrators. Since spike levels ranged from 0 to 30 ng/mL, only the first five calibrators (A-E) were used for these derivations. These calibrators have a range of 0 to 41 ng/mL and were verified by the Elecsys total PSA test. FIG. 4 shows a calibration dose-response curve. The data are shown in Table II, below.

TABLE II

| | Elecsys | Elecsys | ORI- | ORI- | | Elecsys |
|---|---|---|---|---|---|---|
| | tPSA | PSA | GEN | GEN | | Experi- |
| Sample | [ng/mL] | Demo | Wet | Dry | M1 | mental |
| Cal A | 0.008 | 0.000 | 0.000 | 0.000 | 0.000 | 0.000 |
| Cal B | 0.361 | 0.328 | 0.389 | 0.419 | 0.405 | 0.357 |
| Cal C | 1.79 | 1.69 | 2.00 | 2.02 | 2.00 | 1.75 |
| Cal D | 8.44 | 8.14 | 9.16 | 9.52 | 9.05 | 8.53 |
| Cal E | 41.36 | 41.42 | 41.20 | 41.13 | 41.23 | 41.34 |
| CD 1 | 0.007 | −0.003 | 0.000 | 0.007 | 0.011 | 0.005 |
| CD 2 | 1.53 | 1.48 | 1.97 | 1.94 | 1.86 | 1.56 |
| CD 3 | 5.68 | 5.48 | 7.03 | 6.72 | 6.49 | 5.76 |
| CD 4 | 26.96 | 25.81 | 30.29 | 28.53 | 27.70 | 27.03 |
| Plasma 1 | <0.006 | −0.011 | 0.002 | 0.001 | −0.008 | 0.067 |
| Plasma 2 | 1.48 | 1.26 | 1.54 | 1.53 | 1.42 | 1.43 |
| Plasma 3 | 5.40 | 4.83 | 5.48 | 5.42 | 4.96 | 5.06 |
| Plasma 4 | 26.68 | 22.80 | 25.63 | 25.72 | 24.28 | 24.69 |
| WB 1 | ND | ND | 0.023 | 0.010 | −0.007 | ND |
| WB 2 | ND | ND | 0.671 | 0.496 | 0.503 | ND |
| WB 3 | ND | ND | 2.49 | 2.23 | 1.28 | ND |
| WB 4 | ND | ND | 10.58 | 10.62 | 6.06 | ND |
| Bio Rad 1 | 0.6305 | 0.55 | 0.661 | 0.635 | 0.414 | 0.539 |
| Bio Rad 2 | 3.61 | 3.199 | 3.827 | 3.587 | 2.456 | 3.063 |
| Bio Rad 3 | 20.315 | 18.316 | 22.259 | 20.456 | 14.402 | 17.546 |

Cal—Calibrator
CD—Calibrator Diluent
WB—Whole Blood
ND—Not Determined

The equations used to derive the concentrations in Table II are shown below:
Elecsys® Experimental: y=20743x+4112; R²=1;
Elecsys® PSA Demo: y=10684x+623; R²=0.9999;
M1: y=7116.8x+328; R²=0.9996
ORIGEN® Dry: y=5560.2x+414; R²=0.999
ORIGEN® Wet: y=5085.3x+410; R²=0.9995

### Example 8

### Percent Recoveries from Whole Blood and Plasma Samples

Percent recoveries from spiked whole blood and plasma samples were calculated as the percentage relative to the amount of PSA present in the calibrator diluent spikes. For plasma, recoveries ranged from 76.2% to 98.9%. Data generated with the Elecsys 1010 consistently showed the highest recoveries with an average of 96.9%, while the ORIGEN and Ml Analyzers had an average recovery of approximately 82% for plasma. Whole blood spike recoveries were much less with a range of 24.9% to 41.3%. Data are shown in Table III, below.

TABLE III

| | | Elecsys | ORI- | ORI- | | Ex- |
|---|---|---|---|---|---|---|
| | Elecsys | PSA | GEN | GEN | | peri- |
| Sample | tPSA | Demo | Wet | Dry | M1 | mental |
| Plasma 2 | 96.7% | 85.0% | 78.5% | 78.6% | 76.2% | 91.8% |
| Plasma 3 | 95.0% | 88.1% | 77.9% | 80.7% | 76.4% | 87.8% |
| Plasma 4 | 98.9% | 88.4% | 84.6% | 90.2% | 87.7% | 91.3% |
| WB 2 | ND | ND | 34.1% | 32.5% | 35.4% | ND |
| WB 3 | ND | ND | 35.3% | 41.1% | 25.8% | ND |
| WB 4 | ND | ND | 34.9% | 41.3% | 24.9% | ND |

WB—Whole Blood
ND—Not Determined

### Example 9

### Results from Lyophilized PSA Calibrators

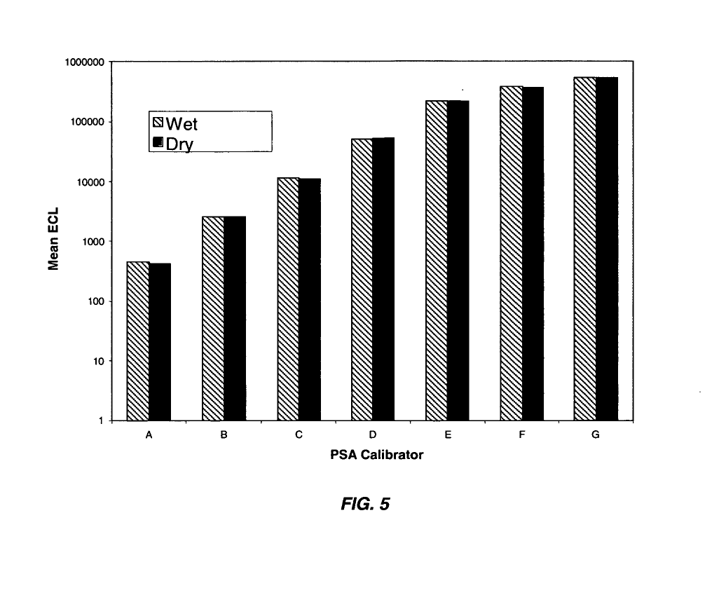

Positive calibrators A-G were each lyophilized with assay reagents in a single tube. These were compared to lyophilized assay reagents to which liquid calibrators were added. As shown in FIG. 5, very good results were obtained for PSA calibrators that were lyophilized together with assay reagents in the same tube when compared to the liquid calibrators.

Lyophilizing PSA calibrators with assay reagents provides an easy to use format since these tubes only require a one step rehydration. In these tubes, two distinct lyophilized pellets were apparent. One pellet was the lyophilized reagent; the other was the lyophilized calibrator. Because the two pellets are distinct, the binding reaction of positive calibrator and assay reagents is not initiated until the assay reagents and positive calibrators are combined during rehydration with either sample or buffer.

These Examples demonstrate that the results for lyophilized PSA calibrators were very similar when compared with tubes that received the calibrator in a liquid.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A dry composition for use as an assay positive control/calibrator comprising:
(a) a labeled binding partner comprising a label and a binding partner wherein said labeled binding partner can specifically bind to an analyte; and
(b) a positive control/calibrator reagent comprising a known amount of the analyte or an analog of the analyte that can specifically bind to the binding partner;
wherein the composition has a moisture content of less than or equal to about 5% by weight, relative to the total weight of the composition.

2. A dry composition for use as an assay positive control/calibrator comprising:
(a) a first binding partner for specifically binding an analyte;
(b) a support for binding the first binding partner without blocking the binding of the analyte;
(c) a labeled second binding partner comprising a label and a binding partner, wherein said labeled second binding partner can specifically bind to the analyte; and
(b) a positive control/calibrator reagent comprising a known amount of the analyte or an analog of the analyte that can specifically bind to both the first binding partner and the second binding partner;
wherein the composition has a moisture content of less than or equal to about 5% by weight, relative to the total weight of the composition.

3. The composition according to claim 2, wherein the first and second binding partners are present in substantially equivalent amounts.

4. A dry composition for use as an assay positive control/calibrator comprising:
(a) a first binding partner for specifically binding an analyte;
(b) a labeled analyte or analog of the analyte comprising a label and an analyte or analog of the analyte, wherein said labeled analyte or analog of the analyte competes with the analyte in a sample for binding to the first binding partner; and
(c) a positive control/calibrator reagent comprising a known amount of the analyte or an analog of the analyte that can specifically bind to the first binding partner;
wherein the composition has a moisture content of less than or equal to about 5% by weight, relative to the total weight of the composition.

5. A dry composition for use as an assay positive control/calibrator comprising:
(a) a first binding partner for specifically binding an analyte;
(b) a support that binds to the first binding partner without blocking the binding of the analyte.
(c) a labeled analyte or analog of the analyte comprising a label and an analyte or analog of the analyte, wherein said labeled analyte or analog of the analyte competes with the analyte in a sample for binding to the first binding partner; and
(d) a positive control/calibrator reagent comprising a known amount of the analyte or an analog of the analyte that can specifically bind to the first binding partner;
wherein the composition has a moisture content of less than or equal to about 5% by weight, relative to the total weight of the composition.

6. The composition of any one of claims 1, 2, 4, or 5, wherein the composition has a moisture content of less than or equal to about 3% by weight, relative to the total weight of the composition.

7. The composition of any one of claims 1, 2, 4, or 5, wherein the composition has a moisture content ranging from about 1% to about 3% by weight, relative to the total weight of the composition.

8. The composition of any one of claims 1, 2, 4, or 5, wherein the composition is lyophilized.

9. The composition of claim 6, wherein the composition is lyophilized.

10. The composition of claim 7, wherein the composition is lyophilized.

11. The composition according to claim 2 or 5, wherein the support is chosen from membranes, beads, particles, electrodes, and surfaces of a container.

12. The composition according to claim 11 wherein the support is a magnetizable bead.

13. The composition according to claim 2 or 5, wherein the support comprises a material chosen from nitrocellulose, polystyrene, polypropylene, carbon, carbon black, EVA, or polyvinyl chloride.

14. The composition according to claim 2 or 5, wherein the support is bound to the first binding partner by a binding pair.

15. The composition according to claim 14, wherein the support is bound to one member of the binding pair and the other member of the binding pair is bound to the first binding partner.

16. The composition according to claim 14, wherein the binding pair is selected from the group consisting of streptavidin/biotin and avidin/biotin.

17. The composition according to claim 2 or 5, wherein the first binding partner is covalently bound to the support.

18. The composition of any one of claims 1, 2 or 5, wherein the label is chosen from radioactive isotopes, enzymes, horseradish peroxidase, and fluorescent groups.

19. The composition of any one of claims 1, 2 or 5, wherein the label is chosen from electrochemiluminescent groups.

20. The composition according to claim 19, wherein the electrochemiluminescent group comprises a metal.

21. The composition according to claim 20, wherein the metal is ruthenium or osmium.

22. The composition according to claim 21, wherein the electrochemiluminescent group is a tris-bipyridyl-ruthenium group.

23. The composition of any one of claims 1, 2, 4, or 5, wherein said known amount of analyte or said analog of the analyte is substantially unbound to any of the binding partners.

24. The composition of any one of claims 1, 2, 4, or 5, wherein the composition comprises an intimate physical mixture of the labeled binding partner and the positive control/calibrator reagent.

25. The composition of any one of claims 1, 2, 4, or 5, wherein the composition comprises at least two adjoining regions in physical contact, wherein at least one first region comprises the labeled binding partner and at least one second region comprises the positive control/calibrator reagent.

26. The composition according to claim 4 or 5, wherein the composition is a control/calibrator composition for performing a competitive binding assay.

27. A method for detecting and/or quantifying an analyte, comprising:
(a) providing a sample, which may contain the analyte;
(b) forming a test reaction mixture by combining the sample with a composition comprising:
(i) a labeled binding partner for specifically binding to the analyte;
(c) forming at least one positive control/calibrator reaction mixture by rehydrating at least one dry composition comprising:
(i) said labeled binding partner; and
(ii) a control/calibrator reagent comprising a known amount of the analyte or an analog of the analyte that can specifically bind to the binding partner;
(d) incubating the test reaction mixture and the at least one positive control/calibrator reaction mixture; and
(e) measuring a signal attributable to a complex formed by binding the analyte to the labeled binding partner for each of the reaction mixtures.

28. The method according to claim 27, further comprising:
(f) assessing the measured signal from (e) by performing at least one step selected from:
(i) confirming successful measurement of the sample; and
(ii) converting the signal generated from the test reaction mixture into a concentration of the test analyte.

29. A method for detecting and/or quantifying an analyte, comprising:
(a) providing a sample, which may contain the analyte;
(b) forming a test reaction mixture by combining the sample with a composition comprising:
(i) a first binding partner for specifically binding the analyte;
(ii) a support for binding the first binding partner without blocking the binding of the analyte; and
(iii) a labeled second binding partner for specifically binding to the same analyte;
(c) forming at least one positive control/calibrator reaction mixture by rehydrating at least one dry composition comprising:
(i) said first binding partner;
(ii) said support;
(iii) said labeled second binding partner; and
(iv) a positive control/calibrator reagent comprising a known amount of the analyte or an analog of the analyte that can specifically bind to both the first binding partner and the second binding partner;
(d) incubating the test reaction mixture and the at least one positive control/calibrator reaction mixture; and
(e) measuring a signal attributable to a complex formed by binding the analyte to the labeled binding partner for each of the reaction mixtures.

30. The method according to claim 29, further comprising:
(f) assessing the measured signal from (e) by performing at least one step selected from:
(i) confirming successful measurement of the sample; and
(ii) converting the signal generated from the test reaction mixture into a concentration of the test analyte.

31. The method according to claim 29 wherein the method is a sandwich binding assay method.

32. A method for detecting and/or quantifying an analyte, comprising:
(a) providing a sample, which may contain the analyte;
(b) forming a test reaction mixture by combining the sample with a composition comprising:
(i) a first binding partner for specifically binding the analyte;
(ii) a labeled analyte or analog of the analyte that competes with the analyte in the sample for binding to the first binding partner;
(c) forming at least one positive control/calibrator reaction mixture by rehydrating at least one dry composition comprising:
(i) said first binding partner;
(ii) said labeled analyte or analog of the analyte; and
(iii) at least one positive control/calibrator reagent comprising a known amount of the analyte or an analog of the analyte that can specifically bind to the first binding partner;
(d) incubating the test reaction mixture and the at least one positive control/calibrator reaction mixture; and
(e) measuring a signal attributable to a complex formed by binding the analyte to said first binding partner for each of the reaction mixtures.

33. The method according to claim 32, wherein at least one positive control/calibrator reaction mixture in (c) is formed by combining the test reaction mixture of (b) with the at least one dry composition.

34. The method according to claim 32, further comprising:
(f) assessing the measured signal from(e) by performing at least one step selected from:
(i) confirming successful measurement of the sample; and
(ii) converting the signal generated from the test reaction mixture into a concentration of the test analyte.

35. A method for detecting and/or quantifying an analyte, comprising:
(a) providing a sample, which may contain the analyte;
(b) forming a test reaction mixture by combining the sample with a composition comprising:
(i) a first binding partner for specifically binding the analyte;
(ii) a support for binding the first binding partner without blocking the binding of the analyte
(iii) a labeled analyte or analog of the analyte that competes with the analyte in the sample for binding to the first binding partner;
(c) forming at least one positive control/calibrator reaction mixture by rehydrating at least one dry composition comprising:
(i) said first binding partner;
(ii) said support
(iii) said labeled analyte or analog of the analyte; and
(iv) at least one positive control/calibrator reagent comprising a known amount of the analyte or an analog of the analyte that can specifically bind to the first binding partner;
(d) incubating the test reaction mixture and the at least one positive control/calibrator reaction mixture; and
(e) measuring a signal attributable to a complex formed by binding the analyte to said first binding partner for each of the reaction mixtures.

36. The method according to claim 35, wherein at least one positive control/calibrator reaction mixture in (c) is formed by combining the test reaction mixture of (b) with the at least one dry composition.

37. The method according to claim 35, further comprising:
(f) assessing the measured signal from(e) by performing at least one step selected from::
(i) confirming successful measurement of the sample; and
(ii) converting the signal generated from the test reaction mixture into a concentration of the test analyte.

38. The method of any one of claims 27, 28, or 32-37, wherein the composition in (b) is a dry composition.

39. The method of any one of claims 27-37, wherein the sample comprises a biological fluid.

40. The method of claim 39, wherein the biological fluid is chosen from blood, plasma, serum, sputum, and saliva.

41. The method of any one of claims 27-37, wherein the sample comprises a naturally occurring body of water.

42. The method of any one of claims 27-37, wherein the sample may contain analytes filtered out of air.

43. The method of any one of claims 27-37, wherein the sample further comprises an aqueous buffer.

44. The method of any one of claims 29, 30, 35, or 37, wherein the method is a competitive binding assay method.

45. The method of any one of claims 27, 29, 30, 35, or 37, wherein the incubating in (d) occurs for a time ranging from about 1 min to about 60 min.

46. The method of any one of claims 27, 29, 30, 35, or 37, wherein the incubating in (d) occurs for a time ranging from about 1 min to about 15 min.

47. The method of any one of claims 27, 29, 30, 35, or 37, wherein the incubating in (d) is performed at a temperature ranging from greater than about 0° C. to about 50° C.

48. The method of any one of claims 27, 29, 30, 35, or 37, wherein the incubating in (d) is performed at about room temperature.

49. The method of any one of claims 27, 29, 30, 35, or 37, wherein the incubating in (d) is performed at about 37° C.

50. The method of any one of claims 27, 29, 30, 35, or 37, wherein the incubating in (d) is performed with a heater.

51. The method of any one of claims 27, 29, 30, 35, or 37, wherein the incubating in (d) is performed with agitation.

52. The method of any one of claims 29, 30, 35, or 37, wherein the second binding partner is labeled with an electrochemiluminescent group and the signal is measured in (e) by a light detector.

53. The method of any one of claims 27, 29, 30, 35, or 37, wherein the signal is at least one of intensity, amplitude, and duration.

54. The method of any one of claims 27, 29, 30, 35, or 37, wherein the method is an electrochemiluminescence assay, the method further comprising adding an assay buffer comprising tripropylamine to the incubated reaction mixture prior to the measuring in (e).

55. The method of any one of claims 27, 29, 30, 35, or 37, wherein the liquid that rehydrates the at least one dry composition for the at least one positive control/calibration reaction mixture is the sample.

56. The method of claim 55 further comprising the step of comparing the signal levels generated from the reaction mixtures to determine if the analyte concentration in the same is sufficiently high that a high-dose hook effect has occurred.

57. A kit comprising
(a) at least one dry composition comprising reagents used for a binding assay and a positive control/calibrator reagent;
(b) at least one container in which each dry composition is located; and
(c) calibration/control information, or a key for obtaining calibration/control information.

58. The kit of claim 57, wherein the reagents used for a binding assay comprises a labeled binding partner comprising a label and a binding partner, wherein the labeled binding partner can specifically bind to an analyte.

59. The kit of claim 58, wherein the at least one dry composition comprises at least first and second dry compositions comprising first and second binding partners, respectively.

60. The kit of claim 59, further comprising a support for binding the first binding partner without blocking the binding of the analyte wherein the first binding partner does not comprise the label.

61. The kit of claim 60, further comprising at least one negative control/calibrator composition comprising at least one dry composition comprising the labeled binding partner, the support, and a third binding partner that
(a) does not specifically bind the analyte;
(b) binds to a support; and
(c) is configured to have similar non-specific binding properties as the first binding partner.

62. The kit according to claim 57,
wherein the number of dry compositions (a) that have distinct known amounts of the analyte or an analog of the analyte is p, p>1;
(e) wherein the analyte has a measurable concentration ranging from c1 to c2, wherein c1<c2
(f) further comprising a second dry composition comprising reagents used for a binding assay without the positive control/calibrator reagent;
wherein when rehydrated by a reagent lacking the analyte, the p known amounts create calibration concentrations of d₁, d₂, . . . , dₚ and wherein d₁<d₂ . . . <dₚ;
wherein the maximum of (i) d₁/c1, (ii) dₘ₊₁/dₘ for 1≦m≦p−1, and (iii) c2/dₚ is less than or equal to about α;
wherein α=2 (c2/((p−1)×c1)) if p>1 and 2 (c2/c1) if p=1.

63. The kit of claim 62, wherein α=2 (c2/(p×c1)).

64. The kit of claim 62, wherein α=2 (c2/(p+1)c1)).

65. The kit of any one of claims 62-64, wherein p=1;

66. The kit of any one of claims 62-64, wherein p=2;

67. The kit of any one of claims 62-64, wherein p=3;

68. The kit of any one of claims 62-64, wherein p=4;

69. The kit according to any one of claims 57-64, wherein the dry composition is the composition of any one of claims 1, 2, 4, or 5.

70. The kit, according to claim 57, wherein
(a) the number of positive control/calibrators with distinct knows amounts of the analyte or an analog of the analyte is p, p≧3;
(b) the p known amounts create calibration concentrations when rehydrated by a reagent lacking the analyte of d₁, d₂, . . . , dₚ and wherein d₁<d₂ . . . <dₚ;
(c) the maximum of (i) (dₘ dₘ₊₂)/(dₘ₊₁ dₘ₊₁) for 1≦m≦p−2 and (ii) (dₘ₊₁ dₘ₊₁)/(dₘ dₘ₊₂) for 1≦m≦p−2 is less than or equal to about γ; and
(d) γ=100.

71. The kit of claim 70, wherein γ=10.

72. The kit of claim 70, wherein γ=2.

73. The kit of claim 70, further comprising: a third binding partner that
(a) does not specifically bind the analyte;
(b) does bind to the support; and
(c) is configured to have similar non-specific binding properties as the first binding partner;
wherein a reaction using the third binding partner in lieu of the first binding partner serves as a negative control/calibrator.

74. The kit according to any one of claims 57-64, wherein said at least one container is at least one well of a multi-well plate.

75. The kit according to any one of claims 57-64, wherein said at least one container is a tube that is less than or equal to about 9 mm in diameter and less than or equal to about 40 mm in height.

76. The kit according to any one of claims 57-64, further comprising a moisture barrier that keeps said dry composition dry for at least 1 month at 25° C. and 100% relative humidity.

77. The kit according to any one of claims 57-64, further comprising a moisture barrier that keeps said dry composition dry for at least 3 months at 45° C. and 100% relative humidity.

78. The kit according to any one of claims 57-64 further comprising two moisture barriers and a desiccant, wherein the desiccant is located between the two moisture barriers.

79. The kit according to any one of claims 57-64, further comprising a humidity indicator, wherein the humidity indicator is located between said two moisture barriers and wherein said humidity indicator is visible from the outside of said two moisture barriers.

80. The kit of claim 57, further comprising a multi-well plate.

81. The kit of claim 57, further comprising a tube that is less than or equal to about 9 mm in diameter and less than or equal to about 40 mm in height.

82. The kit of claim 81, wherein said label comprises ruthenium.

83. The kit of claim 57, further comprising a moisture barrier that keeps the dry composition dry for at least 1 month at 25° C. and 100% relative humidity.

84. The kit of claim 83, wherein the dry composition has a moisture content of less than or equal to about 5% by weight, relative to the total weight of the composition.

85. The kit of claim 57, further comprising a moisture barrier that keeps the dry composition dry for at least 1 month at 45° C. and 100% relative humidity

86. The kit of claim 85, wherein the dry composition has a moisture content of less than or equal to about 5% by weight, relative to the total weight of the composition.

87. The kit according to any one of claims 57-64, wherein said label is an electrochemiluminescent moiety.

88. The kit according to any one of claims 57-64, wherein the electrochemiluminescent moiety comprises a tris-bipyridyl-ruthenium group

89. The kit according to any one of claims 57-64, wherein said dry composition is the composition of claim 2 or 5, and said support is a magnetizable bead.

90. The kit according to claim 57, further comprising instructions for performing a sandwich binding assay.

91. The Kit according to claim 57, further comprising instructions for performing an electrochemiluminescence assay.

92. The kit according to claim 91, further comprising tripropylamine.

93. The kit according to claim 57, further comprising instructions for performing a competitive binding assay.

94. A method of preparing a composition, comprising:
(a) preparing a first solution comprising a labeled binding partner;
(b) freezing the solution formed in (a);
(c) adding a second solution comprising a positive control/calibrator reagent to the frozen mixture at a temperature sufficient to freeze the second solution; and
(d) drying the first and second solutions.

95. The method of claim 94, wherein the first solution further comprises a second binding partner and, optionally, a support that may or may not be pre-associated with the second binding partner.

96. The method according to claim 95, wherein the composition is a control/calibrator composition for performing a sandwich binding assay.

97. A method of preparing a composition, comprising:
(a) preparing a first solution comprising an assay buffer and a first binding partner for specifically binding an analyte;
(b) freezing the mixture formed in (a);
(c) adding a second solution to the frozen mixture at a temperature sufficient to freeze the second solution, said second solution comprising a positive control/calibrator reagent and a labeled analyte or analog of the analyte that competes with the analyte in a sample for binding to the binding partner, and (d) drying the first and second solution

98. The method of claim 97, where said first solution further comprises a support that binds to the first binding partner without blocking the binding of the analyte

99. The method according to any one of claims 94, 95, 97, or 98, wherein the temperature at which the second solution freezes in (c) is sufficiently low to prevent reaction or binding between the binding partners and the at least one positive control/calibrator reagent.

100. A method of preparing a composition, comprising:
(a) drying a first solution comprising a labeled binding partner;
(b) drying a second solution comprising a positive control/calibrator reagent; and
(c) combining the dried first and second solutions.

101. The method of claim 100, wherein the first solution further comprises a second binding partner and, optionally, a support that may or may not be pre-associated with the second binding partner

102. The method according to claim 101, wherein the composition is a control/calibrator composition for performing a sandwich binding assay.

103. A method of preparing a composition, comprising:
(a) drying a first solution comprising a first binding partner for specifically binding an analyte and an assay buffer;
(b) drying a second solution comprising (i) a positive control/calibrator reagent and (ii) a labeled analyte or analog of the analyte that competes with the analyte in a sample for binding to the binding partner; and
(c) combining the dried first and second solutions.

104. The method of claim 103, where said first solution further comprises a support that binds to the first binding partner without blocking the binding of the analyte

105. The method according to any one of claims 94-98 or 100-104, wherein the first solution is prepared in at least one well of a multi-well plate.

106. The method according to any one of claims 94-98 or 100-104, wherein the first solution is prepared in a tube that is less than or equal to about 9 mm in diameter and less than or equal to about 40 mm in height.

107. The method according to any one of claims 95, 98, 101, or 104, wherein said support and said first binding partner are bound together.

108. The method according to any one of claims 94, 95, 98, 100, 101, 103, or 104, wherein the drying is performed by lyophilization.

109. The method according to any one of claims 94, 95 98, 100, 101, 103, or 104, wherein prior to the drying, the combined first and second solutions further comprise a lyophilization buffer.

110. The method according to any one of claims 95, 98, 101, or 104, wherein the support is treated to block or reduce the nonspecific binding of the labeled second binding partner, of the labeled analyte, or of the labeled analog of the analyte to the support.

111. The method according to claim 110, wherein the at least one blocking agent is chosen from goat serum, bovine serum albumin, biotin, and milk proteins.

112. A composition prepared by the method according to any one of claims 94, 95, 97, 98, 100, 101, 103, or 104.

113. A dry composition for use as an assay positive control/calibrator comprising:
(a) at least one labeled binding partner, each labeled binding partner comprising a label and a binding partner wherein said labeled binding partner can specifically bind to at least one analyte; and
(b) a positive control/calibrator reagent comprising a known amount of the at least one analyte or an analog thereof that can specifically bind to the binding partner;
wherein the composition has a moisture content of less than or equal to about 5% by weight, relative to the total weight of the composition.

114. The composition according to claim 113, wherein the positive control/calibrator reagent comprises two or more positive control/calibrator reagents, each positive control/calibrator reagent containing a known amount of one of the at least one analyte or an analog thereof.

115. A dry composition for use as an assay positive control/calibrator comprising:
(a) a first binding partner for specifically binding at least one analyte;
(b) a support for binding the first binding partner without blocking the binding of the at least one analyte;
(c) a labeled second binding partner comprising a label and a binding partner, wherein said labeled second binding partner can specifically bind to the at least one analyte; and
(b) a positive control/calibrator reagent comprising a known amount of the at least one analyte or an analog thereof that can specifically bind to both the first binding partner and the second binding partner;
wherein the composition has a moisture content of less than or equal to about 5% by weight, relative to the total weight of the composition.

116. A kit comprising
(a) at least one dry composition, each dry composition comprising reagents used for a binding assay and a positive control/calibrator reagent;
(b) at least one container in which the at least one dry composition is located; and
(c) calibration/control information, or a key for obtaining calibration/control information.

117. The kit according to claim 116, wherein the at least one dry composition comprises two or more dry compositions, and the at least one container comprises two or more containers, each container containing one of the two or more dry compositions.

118. The kit according to claim 116, wherein the reagents used for a binding assay comprises a labeled binding partner comprising a label and a binding partner, wherein the labeled binding partner can specifically bind to at least one analyte, and the at least one positive control/calibrator reagent comprises two or more positive control/calibrator reagents, each reagent comprising a known amount of one of the at least one analyte.

119. A method for detecting and/or quantifying at least one analyte, comprising:
(a) providing a sample, which may contain the at least one analyte;
(b) forming a test reaction mixture by combining the sample with at least one composition, each composition comprising:
(i) a labeled binding partner for specifically binding to the at least one analyte;
(c) forming at least one positive control/calibrator reaction mixture by rehydrating at least one dry composition, each dry composition comprising:
(i) said labeled binding partner; and
(ii) a control/calibrator reagent comprising a known amount of one of the at least one analyte or an analog thereof that can specifically bind to the binding partner;
(d) incubating the test reaction mixture and the at least one positive control/calibrator reaction mixture; and
(e) measuring a signal attributable to a complex formed by binding the one of the at least one analyte to the labeled binding partner for each of the reaction mixtures.

120. The method according to claim 119, wherein at least one positive control/calibrator reaction mixture in (c) is formed by combining the test reaction mixture of (b) with the at least one dry composition.

121. A method for detecting and/or quantifying at least one analyte, comprising:
(a) providing a sample, which may contain the at least one analyte;
(b) forming a test reaction mixture by combining the sample with at least one composition, each composition comprising:
(i) a first binding partner for specifically binding one of the at least one analyte;
(ii) a support for binding the first binding partner without blocking the binding of the one analyte; and
(iii) a labeled second binding partner for specifically binding to the same analyte;
(c) forming at least one positive control/calibrator reaction mixture by rehydrating at least one dry composition comprising:
(i) said first binding partner;
(ii) said support;
(iii) said labeled second binding partner; and
(iv) a positive control/calibrator reagent comprising a known amount of the one analyte or an analog thereof that can specifically bind to both the first binding partner and the second binding partner;
(d) incubating the test reaction mixture and the at least one positive control/calibrator reaction mixture; and
(e) measuring a signal attributable to a complex formed by binding the one analyte to the labeled binding partner for each of the reaction mixtures.

122. A kit comprising at least one dry composition for use as an assay positive control/calibrator comprising:
(a) a labeled binding partner comprising a label and a binding partner wherein said labeled binding partner can specifically bind to an analyte; and
(b) a positive control/calibrator reagent comprising a known amount of the analyte or an analog of the analyte that can specifically bind to the binding partner;
wherein the composition has a moisture content of less than or equal to about 5% by weight, relative to the total weight of the composition, and wherein each of the at least one dry composition is positioned within a container.

123. The kit according to claim 122, wherein the labeled binding partner and positive control/calibrator reagent is positioned within the tube such that they are capable of contacting each other.
